(19) United States
(12) Patent Application Publication
(10) Pub. No.: US 2026/0293897 A1
(43) Pub. Date: Oct. 1, 2026

(54) COMPOUND CONTAINING FIVE-MEMBERED HETEROCYCLE, PREPARATION METHOD THEREFOR, AND APPLICATION THEREOF

(71) Applicant: QINGDAO KANGQIAO PESTICIDES AND CHEMICALS GROUP CO., LTD., Qingdao, Shandong (CN)

(72) Inventors: Xiangwei LIU, Shandong (CN); Yingshuai LIU, Shandong (CN); Qinan HAN, Shandong (CN); Baohong LIU, Shandong (CN); Guozhu SHENG, Shandong (CN); Jiaxing LI, Shandong (CN); Dezhi LI, Shandong (CN); Ying CHEN, Shandong (CN); Ruibin LIU, Shandong (CN); Yaoyao DU, Shandong (CN); Shiling WANG, Shandong (CN); Chen GONG, Shandong (CN); Ying WANG, Shandong (CN); Min LIU, Shandong (CN); Yunxiao SUN, Shandong (CN); Haixia YU, Shandong (CN); Ruijie FENG, Shandong (CN); Shien FAN, Shandong (CN); Yu CHEN, Shandong (CN); Ning LI, Shandong (CN)

(73) Assignee: QINGDAO KANGQIAO PESTICIDES AND CHEMICALS GROUP CO., LTD., Qingdao, Shandong (CN)

(21) Appl. No.: 19/131,404

(22) PCT Filed: Sep. 18, 2023
(86) PCT No.: PCT/CN2023/119499

§ 371 Date: May 20, 2025

(30) Foreign Application Priority Data
Sep. 21, 2022 (CN) 202211151392.2
Sep. 21, 2022 (CN) 202211151575.4

(51) Int. Cl. A01N 43/80; A01P 5/00; A01P 7/02; A01P 7/04; C07D 413/12

(52) U.S. Cl. CPC A01N 43/80; A01P 5/00; A01P 7/02; A01P 7/04; C07D 413/12

(57) ABSTRACT

Disclosed are a compound containing a five-membered heterocycle, a preparation method therefor, an application thereof, an agricultural composition, and a method for preventing and controlling invertebrate pests. The present invention provides a compound containing a five-membered heterocycle having a structure represented by formula (I), or an agriculturally acceptable salt thereof, or a stereoisomer thereof. The compound containing a five-membered heterocycle and the agriculturally acceptable salt thereof provided by the present invention exhibit an excellent prevention and control effect against various pests, especially spider mites such as carmine spider mites, red spider mites, Kanzawa spider mites and citrus spider mites and lepidopteran insects such as the diamondback moths. The compound may be used for preventing and controlling various pests and pest mites, exhibits highly effective acaricidal and insecticidal activity, and has good application prospects.

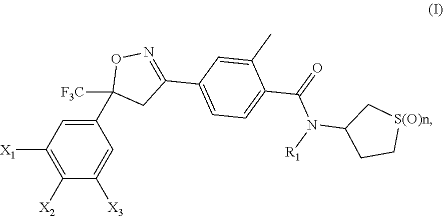

### CROSS REFERENCE TO RELATED APPLICATIONS

This application claims the benefits of Chinese Patent Applications No. 202211151575.4 and No. 202211151392.2, filed on Sep. 21, 2022. The contents of these applications are incorporated herein by reference.

### FIELD

The present invention relates to the technical field of pesticides, specifically to compounds containing five-membered heterocycles, their preparation methods and applications, agricultural compositions, and methods for controlling invertebrate pests.

### BACKGROUND

Pests and mites damage hundreds of plant species, especially vegetables, flowers, and fruit trees, causing serious economic losses. Aryl isoxazole derivatives containing heterocyclic compounds have unique insecticidal activities in modern crop and animal protection. For example, fluxametamide, the first isoxazoline-type insecticide developed and marketed by Nissan Chemical, is such a derivative.

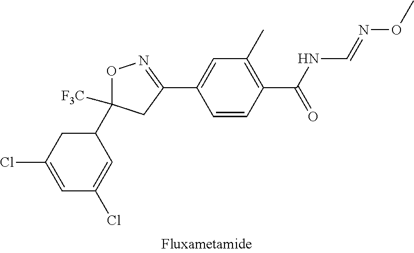 [SMILES: CO/N=C/NC(=O)c1ccc(C2=NOC(C3C=C(Cl)C=C(Cl)C3)(C(F)(F)F)C2)cc1C]

The heterocyclic compound is an important intermediate or chemical entity in the synthesis and development processes of pesticides, has a unique chemical structure and biological activity, and a plurality of derivatives of the heterocyclic compound are developed into agricultural chemical products.

CN106045962A discloses that a compound (x.82) shown in the following structure has over 80% of lethal activity to pests such as Spodoptera litura, Helicoverpa assulta, Plutella xylostella, Phyllotreta striolata, Thrips tabaci, Tetranychus urticae, Myzus persicae, Euschistus heros, and Nilaparvata lugens at a concentration of 200 ppm (200 mg/L), however, the activity to agricultural pests at lower concentration is not reported.

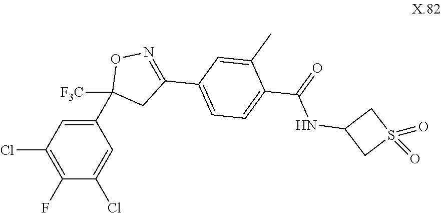 [SMILES: Cc1cc(C2=NOC(c3cc(Cl)c(F)c(Cl)c3)(C(F)(F)F)C2)ccc1C(=O)NC1CS(=O)(=O)C1]

Chlorantraniliprole is a bisamide insecticide developed by DuPont, is the most successful insecticide developed so far and has the largest sales, and particularly has high control effect on lepidoptera pests such as Plutella xylostella and the like. However, as the chlorantraniliprole is continuously used in fields for many years, the resistance of the chlorantraniliprole to the pests such as Plutella xylostella is very serious, the pesticide effect is obviously reduced, and the pesticide with good control effect on the lepidoptera pests such as the resistant Plutella xylostella is urgently needed in the market.

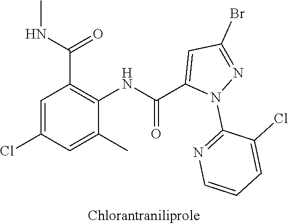 [SMILES: CNC(=O)c1cc(Cl)cc(C)c1NC(=O)c1cc(Br)nn1-c1ncccc1Cl]

The insecticidal and acaricidal effects of the compounds disclosed at present are still unsatisfactory, the resistance of pests and mites develops rapidly, and novel compounds with higher efficiency are still needed in agricultural production to cope with the increasing development of resistance of pests and mites.

### SUMMARY

The invention aims to overcome the technical problem that the existing compounds in the prior art have lower killing activity in the prevention and control of invertebrate pests (Particularly mites and Plutella xylostella), and provides a more efficient compounds containing five-membered heterocycles, their preparation methods and applications, agricultural compositions, and methods for controlling invertebrate pests. The compound containing the five-membered heterocyclic ring provided by the invention has high acaricidal and insecticidal activity, and particularly shows high control effect on Tetranychus cinnabarinus, Plutella xylostella and the like.

In order to achieve the above object, the present invention provides, in a first aspect, a five-membered heterocycle-containing compound having a structure represented by formula (I) or an agriculturally acceptable salt thereof, or a stereoisomer thereof,

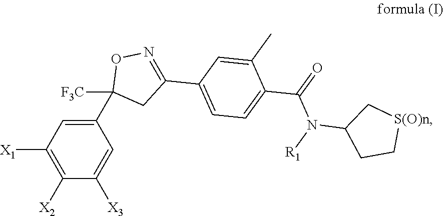

in formula (I),
X₁ and X₃ are each independently F, Cl, Br, I or CF₃;
X₂ is H, F, Cl, Br, I or CF₃;
R₁ is selected from the group consisting of hydrogen, substituted or unsubstituted C₁-C₆ alkyl, C₁-C₄ alkoxy C₁-C₄ alkyl, C₁-C₁₀ alkylcarbonyl, halo C₁-C₁₀ alkylcarbonyl, C₃-C₁₀ cycloalkylcarbonyl, C₃-C₁₀ cycloalkylmethylcarbonyl, C₁-C₁₀ alkoxycarbonyl, halo C₁-C₁₀ alkoxycarbonyl, C₁-C₁₀ alkoxy-C₁-C₄ alkylcarbonyl, halo C₁-C₁₀ alkoxy C₁-C₄ alkylcarbonyl, C₁-C₁₀ alkyl oxalyl, halo C₁-C₁₀ alkyl oxalyl, C₁-C₁₀ alkoxyoxalyl, halo C₁-C₁₀ alkoxyoxalyl, C₁-C₁₀ alkylsulphonyl, halo C₁-C₁₀ alkylsulphonyl, C₁-C₁₀ alkylsulfinyl, halo C₁-C₁₀ alkylsulfinyl, substituted or unsubstituted benzoyl, substituted or unsubstituted phenylsulfonyl, substituted or unsubstituted heteroarylcarbonyl, substituted or unsubstituted C₁-C₆ alkyl, or C₁-C₄ alkoxy C₁-C₄ alkyl;
n is selected from 0, 1 or 2.

A second aspect of the present invention provides a method of preparing a compound containing a five-membered heterocycle, the method comprising:
(1) compound II and compound III are subjected to a first reaction in a first solvent in the presence of a condensing agent, to obtain compound IV;
(2) compound IV and compound V are subjected to a second reaction in a second solvent in the presence of an alkaline substance, to obtain compound I;
wherein the compound II has a structure represented by formula (II), the compound III has a structure represented by formula (III), the compound IV has a structure represented by formula (IV), the compound V has a structure represented by formula (V), and the compound I has a structure shown in a formula (I),

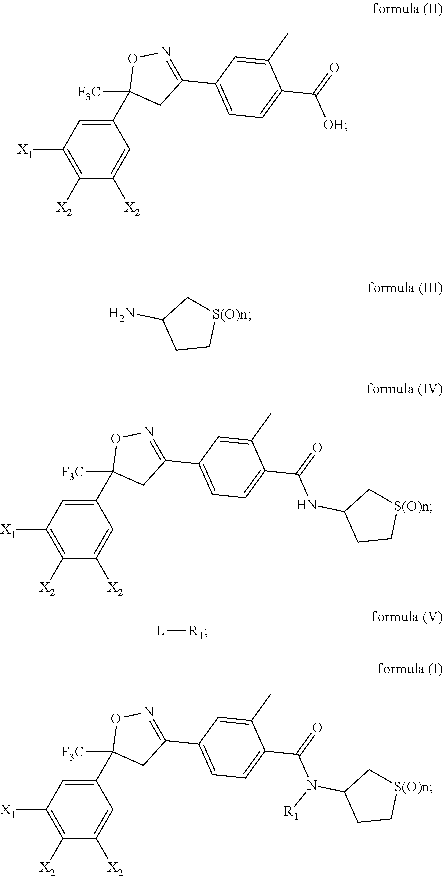

in formula (I), formula (II), formula (III), formula (IV), and formula (V),
X₁, X₂, and X₃ are each independently selected from F, Cl, Br, I or CF₃;
R₁ is selected from the group consisting of hydrogen, substituted or unsubstituted C₁-C₆ alkyl, C₁-C₄alkoxy C₁-C₄ alkyl, C₁-C₁₀ alkylcarbonyl, halo C₁-C₁₀ alkylcarbonyl, C₃-C₁₀ cycloalkylcarbonyl, C₃-C₁₀ cycloalkylmethylcarbonyl, C₁-C₁₀ alkoxycarbonyl, halo C₁-C₁₀ alkoxycarbonyl, C₁-C₁₀ alkoxy-C₁-C₄ alkylcarbonyl, halo C₁-C₁₀ alkoxy C₁-C₄ alkylcarbonyl, C₁-C₁₀ alkyl oxalyl, halo C₁-C₁₀ alkyl oxalyl, C₁-C₁₀ alkoxyoxalyl, halo C₁-C₁₀ alkoxyoxalyl, C₁-C₁₀ alkylsulphonyl, halo C₁-C₁₀ alkylsulphonyl, C₁-C₁₀ alkylsulfinyl, halo C₁-C₁₀ alkylsulfinyl, substituted or unsubstituted benzoyl, substituted or unsubstituted phenylsulfonyl, substituted or unsubstituted heteroarylcarbonyl, substituted or unsubstituted C₁-C₆ alkyl, or C₁-C₄ alkoxy C₁-C₄ alkyl;
L is selected from fluorine, chlorine, bromine, iodine, methylsulfonyl, trifluoromethanesulfonyl, phenylsulfonyl or p-toluenesulfonyl.

In a third aspect, the invention provides the use of a compound as described above or a compound prepared by a process as described above for controlling invertebrate pests.

In a fourth aspect, the present invention provides an agricultural composition comprising at least one of the foregoing compounds or prepared by the foregoing methods and at least one liquid carrier or solid carrier.

In a fifth aspect the invention provides a method of controlling invertebrate pests, said method comprising: applying an insecticidally effective amount of at least one of the foregoing compounds or prepared by the foregoing preparation method, directly or indirectly to an invertebrate pest to be controlled and/or to a medium on which the invertebrate pest grows.

Through the technical scheme, the beneficial technical effects obtained by the invention are as follows:
(1) The compound containing a five-membered heterocycle provided by the present invention and its agriculturally acceptable salts exhibit excellent control effects against various pests, especially leaf mites represented by Tetranychus cinnabarinus, Tetranychus urticae Koch, Tetranychus kanzawai, Panonychus citri Mc Gregor, etc, lepidoptera represented by the Plutella xylostella, Thrips and Flea beetles have shown high control results, have high acaricidal and insecticidal activities, and have good application prospects.
(2) The five-membered heterocycle-containing compounds provided by the present invention have high activity against the following invertebrate pests (the following are intended to illustrate the invention, but not to limit the invention): Tetranychidae (e.g., Tetranychus cinnabarinus, Panonychus citri Mc Gregor, Tetranychus urticae Koch, Panonychus ulmi, Tetranychus kanzawai, Tetranychus viennensis Zacher), Eriophyidae, Eupodoidea, Tenuipalpidae, Aphididae (e.g., Myzus persicae), Lepidoptera (e.g., Plutella xylostella, Spodoptera exigua, Chilo suppressalis, etc.), Thysanoptera (e.g., Frankliniella occidentalis, etc.), Coleoptera (e.g., Phyllotreta striolata), and the like. Therefore, the compound containing the five-membered heterocyclic ring can be applied to preparing insecticide and acaricide medicaments in agriculture or other fields.

### DETAILED DESCRIPTION

The present invention provides, in a first aspect, a five-membered heterocycle-containing compound having a structure represented by formula (I) or an agriculturally acceptable salt thereof, or a stereoisomer thereof,

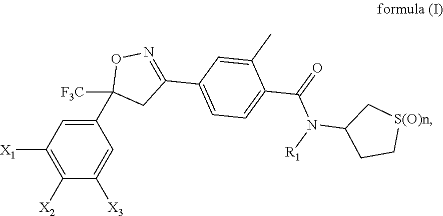

in formula (I),
X₁ and X₃ are each independently F, Cl, Br, I or CF₃;
X₂ is H, F, Cl, Br, I or CF₃;
R₁ is selected from the group consisting of hydrogen, substituted or unsubstituted C₁-C₆ alkyl, C₁-C₄ alkoxy C₁-C₄ alkyl, C₁-C₁₀ alkylcarbonyl, halo C₁-C₁₀ alkylcarbonyl, C₃-C₁₀ cycloalkylcarbonyl, C₃-C₁₀ cycloalkylmethylcarbonyl, C₁-C₁₀ alkoxycarbonyl, halo C₁-C₁₀ alkoxycarbonyl, C₁-C₁₀ alkoxy-C₁-C₄ alkylcarbonyl, halo C₁-C₁₀ alkoxy C₁-C₄ alkylcarbonyl, C₁-C₁₀ alkyl oxalyl, halo C₁-C₁₀ alkyl oxalyl, C₁-C₁₀ alkoxyoxalyl, halo C₁-C₁₀ alkoxyoxalyl, C₁-C₁₀ alkylsulphonyl, halo C₁-C₁₀ alkylsulphonyl, C₁-C₁₀ alkylsulfinyl, halo C₁-C₁₀ alkylsulfinyl, substituted or unsubstituted benzoyl, substituted or unsubstituted phenylsulfonyl, substituted or unsubstituted heteroarylcarbonyl, substituted or unsubstituted C₁-C₆ alkyl, or C₁-C₄ alkoxy C₁-C₄ alkyl.

In some preferred embodiments according to the present invention, when X₂ is H, X₁ and X₃ are not both Cl. More preferably, X₃ is CF₃ when X₂ is H.

In some embodiments, X₁, X₂, and X₃ are each independently selected from F, Cl, Br, or CF₃.

In some embodiments, R₁ is selected from the group consisting of hydrogen, methyl, ethyl, n-propyl, isopropyl, n-butyl, isobutyl, sec-butyl, tert-butyl, methoxymethyl, methoxyethyl, ethoxymethyl, ethoxyethyl, trifluoromethyl, trichloromethyl, trifluoroethyl or trifluoromethoxy methyl, C₁-C₆ alkylcarbonyl, halo C₁-C₆ alkylcarbonyl, C₃-C₆ cycloalkylcarbonyl, C₃-C₆ cycloalkylmethylcarbonyl, C₁-C₆ alkoxycarbonyl, halo C₁-C₆ alkoxycarbonyl, C₁-C₄ alkoxy C₁-C₂ alkylcarbonyl, halo C₁-C₄ alkoxy C₁-C₂ alkylcarbonyl, C₁-C₆ alkoxalyl, halo C₁-C₆ alkoxalyl, C₁-C₄ alkoxyoxalyl, halo C₁-C₄ alkoxyoxalyl, C₁-C₄ alkylsulfonyl, halo C₁-C₄ alkylsulfonyl, C₁-C₄ alkylsulfinyl, halo C₁-C₄ alkylsulfinyl, halogen substituted or unsubstituted benzoyl, halogen substituted or unsubstituted benzenesulfonyl, carbonyl substituted with a five- or six-membered heteroaromatic ring containing 1-2 heteroatoms selected from O, S or N.

In some embodiments, X₁, X₂, and X₃ are each independently selected from F, Cl or CF₃.

In some preferred embodiments, X₂ is selected from F or Cl, and X₁ and X₃ are each independently selected from Cl or CF₃.

In some embodiments, R₁ is selected from the group consisting of hydrogen, methyl, ethyl, n-propyl, isopropyl, n-butyl, isobutyl, sec-butyl, tert-butyl, methoxymethyl, methoxyethyl, ethoxymethyl, ethoxyethyl, acetyl, propionyl, n-butyryl, cyclopropylformyl, methoxyformyl group, methoxyacetyl, methoxyacetyl, methoxy oxalyl, ethoxy oxalyl.

In some embodiments, the compound is selected from at least one of the compounds represented by the following structures, or an agriculturally acceptable salt thereof, or a stereoisomer thereof:

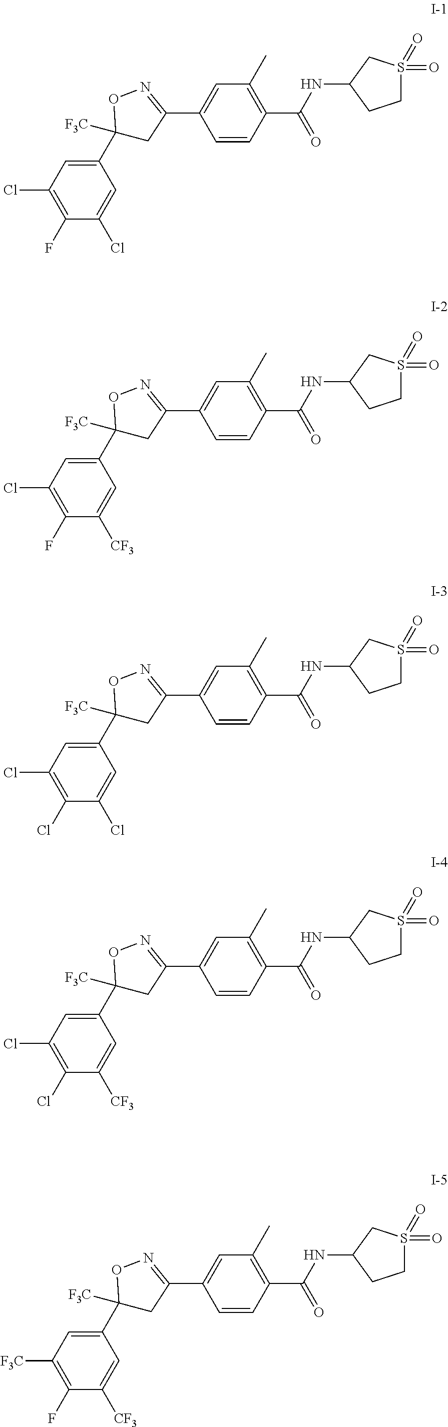

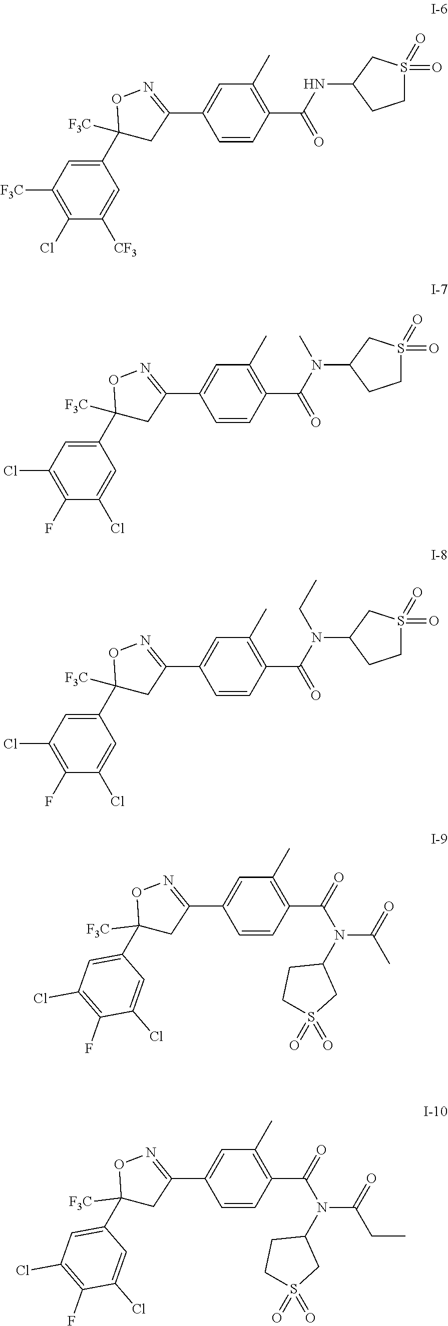

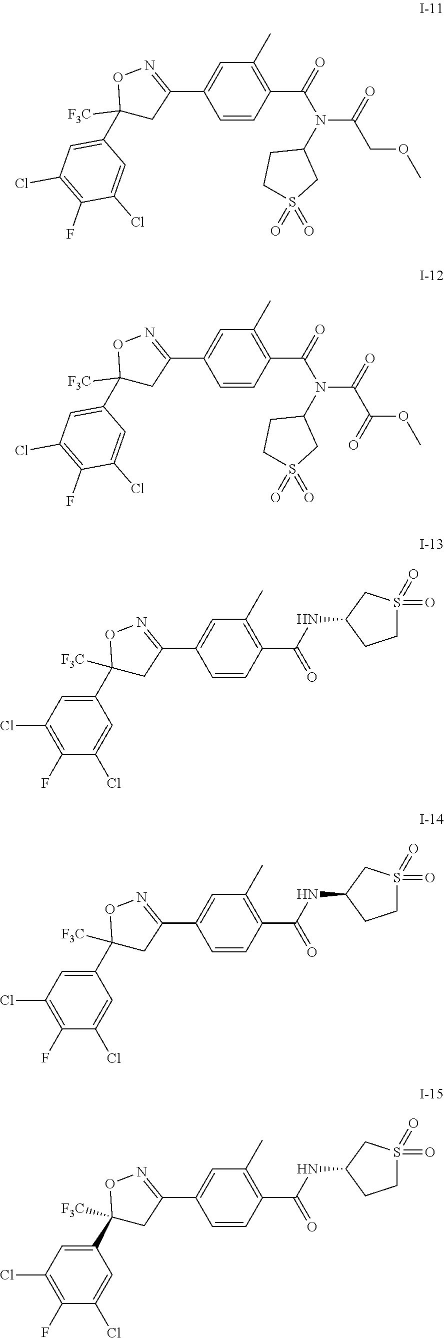

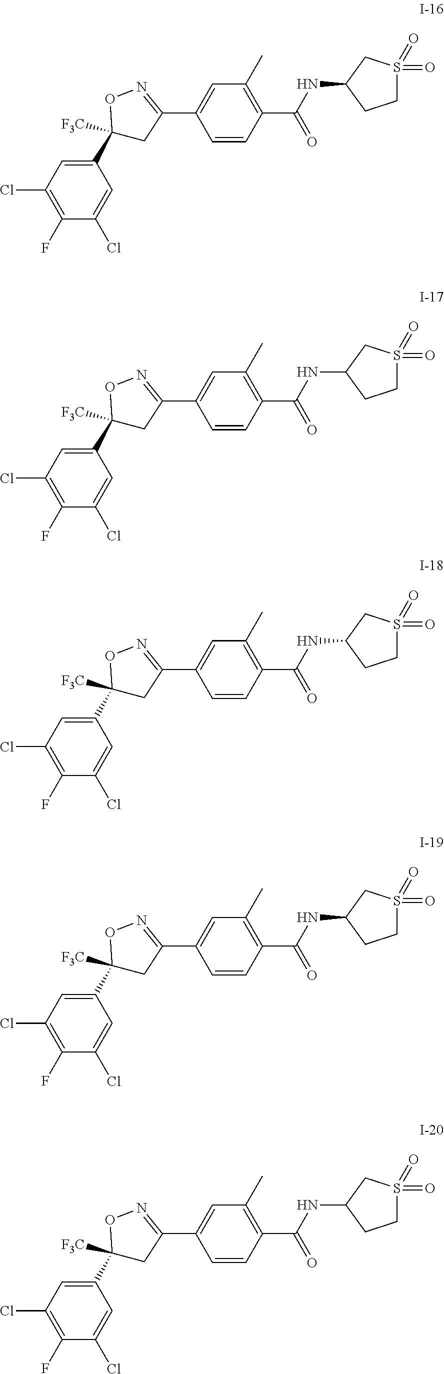

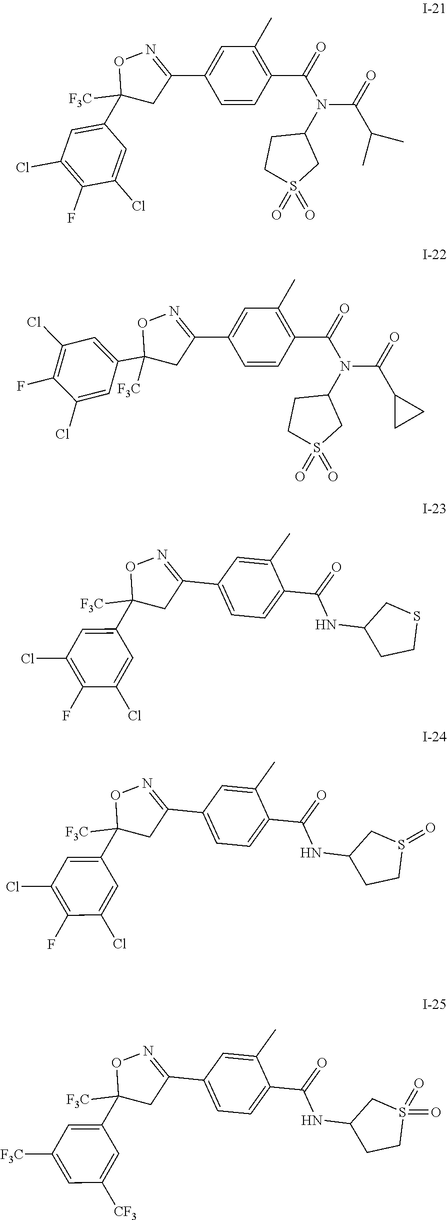

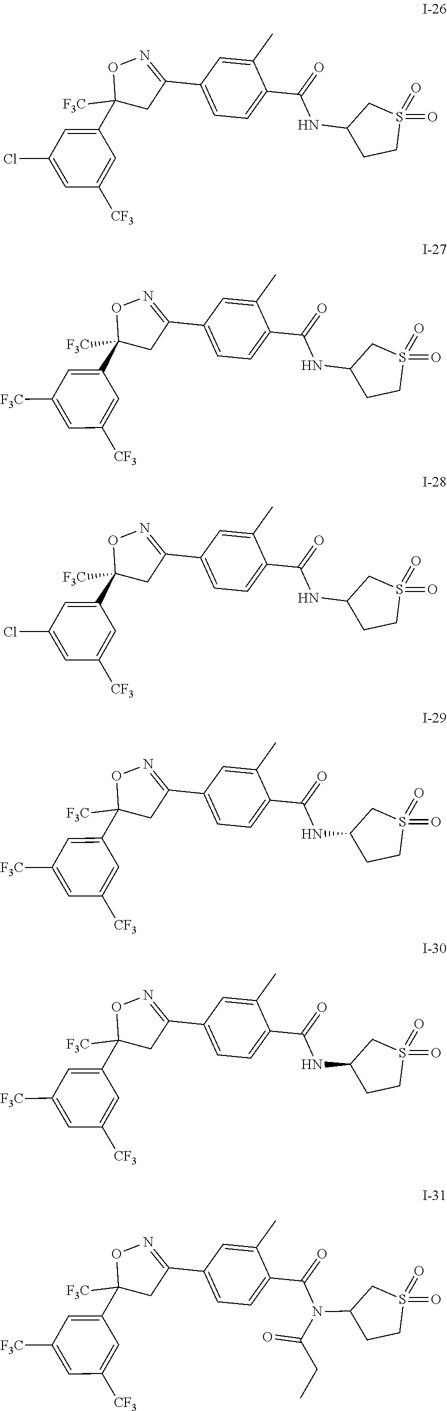

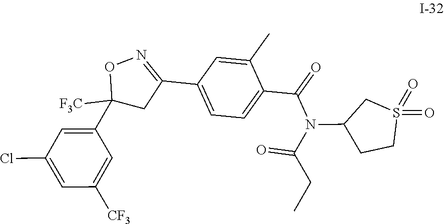

wherein, the compounds I-13, I-14, I-15, I-16, I-17, I-18, I-19 and I-20 are stereoisomers of the compound I-1; compounds I-27, I-29, I-30 are stereoisomers of compounds I-25, and compound I-28 is a stereoisomer of compound I-26.

In the present invention, said five-membered heterocyclic-containing compound exists in the case of optical isomers caused by having 1 or more than 2 asymmetric carbon atoms, and the present invention includes all optical isomers, racemic or diastereomeric.

In the present invention, the five-membered heterocycle-containing compound also includes derivatives thereof, but the compounds included in the derivatives of the present invention are by no means limited to these compounds.

In the present invention, the so-called agriculturally acceptable salt refers to a salt formed by a compound containing a five-membered heterocyclic ring with an inorganic acid or an organic acid. As inorganic acids, for example, hydrochloric acid, sulfuric acid, hydrobromic acid, and the like, and as organic acids, for example, formic acid, acetic acid, methanesulfonic acid, fumaric acid, maleic acid, and the like.

A second aspect of the present invention provides a method of preparing a compound containing a five-membered heterocycle, the method comprising:
(1) compound II and compound III are subjected to a first reaction in a first solvent in the presence of a condensing agent, to obtain compound IV;
(2) compound IV and compound V are subjected to a second reaction in a second solvent in the presence of an alkaline substance, to obtain compound I;
wherein the compound II has a structure represented by formula (II), the compound III has a structure represented by formula (III), the compound IV has a structure represented by formula (IV), the compound V has a structure represented by formula (V), and the compound I has a structure shown in a formula (I),

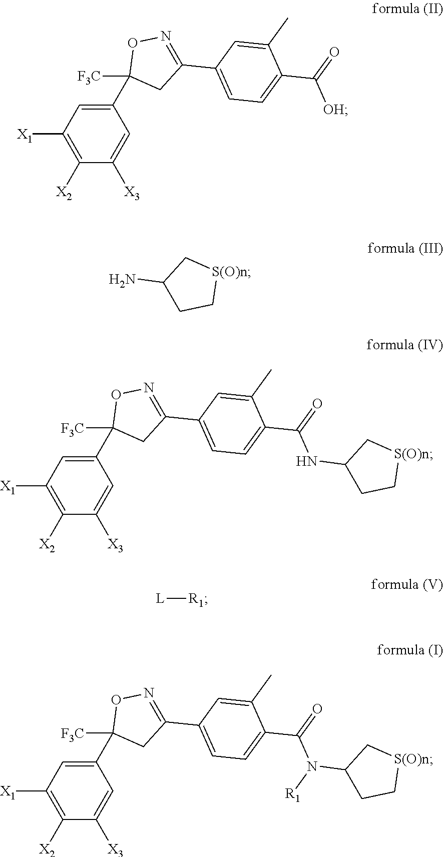

in formula (I), formula (II), formula (III), formula (IV) and formula (V),
X₁, X₂, and X₃ are each independently selected from F, Cl, Br, I or CF₃;
R₁ is selected from the group consisting of hydrogen, substituted or unsubstituted C₁-C₆ alkyl, C₁-C₄alkoxy C₁-C₄ alkyl, C₁-C₁₀ alkylcarbonyl, halo C₁-C₁₀ alkylcarbonyl, C₃-C₁₀ cycloalkylcarbonyl, C₃-C₁₀ cycloalkylmethylcarbonyl, C₁-C₁₀ alkoxycarbonyl, halo C₁-C₁₀ alkoxycarbonyl, C₁-C₁₀ alkoxy-C₁-C₄ alkylcarbonyl, halo C₁-C₁₀ alkoxy C₁-C₄ alkylcarbonyl, C₁-C₁₀ alkyl oxalyl, halo C₁-C₁₀ alkyl oxalyl, C₁-C₁₀ alkoxyoxalyl, halo C₁-C₁₀ alkoxyoxalyl, C₁-C₁₀ alkylsulphonyl, halo C₁-C₁₀ alkylsulphonyl, C₁-C₁₀ alkylsulfinyl, halo C₁-C₁₀ alkylsulfinyl, substituted or unsubstituted benzoyl, substituted or unsubstituted phenylsulfonyl, substituted or unsubstituted heteroarylcarbonyl, substituted or unsubstituted C₁-C₆ alkyl, or C₁-C₄ alkoxy C₁-C₄ alkyl;
L is selected from fluorine, chlorine, bromine, iodine, methylsulfonyl, trifluoromethanesulfonyl, phenylsulfonyl or p-toluenesulfonyl.

In the present invention, the selection of X₁, X₂, X₃ and R₁ is the same as that of the above-mentioned compounds, and the details thereof are not repeated.

In some embodiments, said condensing agent is selected from one or more of dicyclohexylcarbodiimide, 1-(3-dimethylaminopropyl)-3-ethylcarbodiimide hydrochloride, 2-(7-azabenzotriazolyl)-N,N,N′,N′-tetramethyluronium hexafluorophosphate, thionyl chloride, or oxalyl chloride.

In some embodiments, the first solvent and the second solvent are each independently selected from one or more of toluene, xylene, dichloromethane, dichloroethane, acetonitrile, N,N-dimethylformamide (DMF), tetrahydrofuran, and acetone.

In some embodiments, the alkaline substance is selected from one or more of triethylamine, diisopropylethylamine, pyridine, N-methylmorpholine, sodium carbonate, potassium carbonate, sodium hydroxide, potassium hydroxide, sodium bicarbonate, potassium bicarbonate, sodium hydride, and sodium amide.

In some embodiments, the molar ratio of compound II to compound III is 1:0.8 to 1.5.

In some embodiments, the molar ratio of compound IV to compound V is 1:0.7 to 1.5.

In some embodiments, the molar ratio of the compound II to the condensing agent is 1:0.8 to 1.5.

In some embodiments, the molar ratio of compound IV to the alkaline substance is 1:0.7 to 1.5.

In some embodiments, the temperature of the first reaction is from −10° C. to the boiling point of the first solvent, and the time of the reaction is from 10 min to 48 h.

In some preferred embodiments, the temperature of the first reaction is from −10° C. to 210° C., preferably from 0° C. to 150° C., more preferably from 10° C. to 100° C.; the time is 0.5 to 24 hours, preferably 1 to 12 hours, more preferably 2 to 6 hours.

In some embodiments, the temperature of the second reaction is from −10° C. to the boiling point of the second solvent, and the time of the reaction is from 10 min to 48 h.

In some preferred embodiments, the temperature of the second reaction is from −10° C. to 210° C., preferably from 0° C. to 150° C., more preferably from 10° C. to 100° C.; the time is 0.5 to 24 hours, preferably 1 to 12 hours, more preferably 2 to 6 hours.

The inventors of the present invention found that the compounds represented by formula (I) have unexpectedly high acaricidal activity, and therefore, the compounds of the present invention can also be applied to the preparation of acaricides in agriculture or other fields. In particular, it has been found that the compounds of formula (I) have a high activity against the invertebrate pests mentioned below (the following are intended to illustrate the invention, but not to limit the invention): Tetranychidae (e.g., Tetranychus cinnabarinus, Panonychus citri Mc Gregor, Tetranychus urticae Koch, Panonychus ulmi, Tetranychus kanzawai, Tetranychus viennensis Zacher), Eriophyidae, Eupodoidea, Tenuipalpidae, Aphididae (e.g., Myzus persicae), Lepidoptera (e.g., Plutella xylostella, Spodoptera exigua, Chilo suppressalis, etc.), and the like. Therefore, the compound of the invention can also be applied to the preparation of acaricide medicines in agriculture or other fields.

In a third aspect, the invention provides the use of a compound as described above or a compound prepared by a process as described above for controlling invertebrate pests.

In some embodiments, the invertebrate pest is a acarid pest, an insect and/or a nematode.

Preferably, in the present invention, said compounds can be used to protect crops, livestock and the like important in agriculture and horticulture from or with less damage by mites.

In the present invention, in order to obtain the desired effect, in some applications, the amount of said compound varies depending on various factors, such as the compound used, the crop to be pre-protected, the type of pest, the degree of infection, the method of application, the environment in which it is applied, the application dosage form, and other factors.

In the present invention, it is preferred that for certain applications, such as agriculture, one or more other insecticides, acaricides, fungicides, herbicides, plant growth regulators or fertilizers, etc., can be added to the acaricidal compositions of the present invention, thereby providing additional advantages and effects.

In a fourth aspect, the present invention provides an agricultural composition comprising at least one of the foregoing compounds or prepared by the foregoing methods and at least one liquid carrier or solid carrier.

The liquid carrier or the solid carrier is not particularly limited in the present invention as long as the requirements of the present invention can be satisfied. In the composition, preferably, the concentration of the compound containing the five-membered heterocycle is 0.5-35 mg/L. In some embodiments, the concentration of the five-membered heterocycle-containing compound is 1-30 mg/L, and in other embodiments, the concentration of the five-membered heterocycle-containing compound is 10-25 mg/L.

In the invention, the liquid carrier can be one or more of water, various aromatic hydrocarbons, aliphatic hydrocarbons, ketones, ethers and the like, such as toluene, xylene, acetone, cyclohexanone, xylene, benzene, cyclohexane, isopropanol, ethylene glycol, sorbitol, methanol, ethanol, butanol, dimethylformamide, N-methylpyrrolidone, naphthane, engine oil, petroleum ether, cyclohexanone, methyl oleate, methylated soybean oil and the like; solid carriers may include natural or synthetic clays and silicates, and solid carriers suitable for dusts may include naturally occurring rock powders, chalk, quartz, clays, montmorillonite, white carbon black, diatomaceous earth, pumice, gypsum, talc, bentonite, kaolin, china clay and synthetic ground minerals (e.g. micro-dispersed silicate or alumina).

Suitable particle carriers may include crushed and graded natural rocks such as calcite, marble, pumice, seafoam and dolomite and synthetic particles made from powders of organic and inorganic materials.

In the present invention, preferably, the composition may be administered in the form of a formulation. Wherein the compound of claim 1 is dissolved or dispersed as an active ingredient in a carrier or formulated to be more easily dispersed for insecticidal or acaricidal use. For example: the composition can be prepared into wettable powder, water dispersible granules, suspending agents, aqueous emulsion, aqueous agent or emulsifiable concentrate and the like. In the composition, at least one liquid or solid carrier is added, and when necessary, a suitable surfactant may also be added.

Wherein, the surfactant can comprise dodecyl benzene sulfonate, fatty alcohol sulfate, tween, concentrated emulsion, sorbitol polyoxyethylene ether, fatty alcohol polyoxyethylene ether, lignosulfonate, alkyl naphthalene sulfonate and the like.

In a fifth aspect the invention provides a method of controlling invertebrate pests, said method comprising: applying an insecticidally effective amount of at least one of the foregoing compounds or prepared by the foregoing preparation method, directly or indirectly to an invertebrate pest to be controlled and/or to a medium on which the invertebrate pest grows.

The present invention is not particularly limited in direct or indirect manner as long as the purpose of controlling invertebrate pests can be achieved, and for example, the direct manner may include directly contacting a substance containing the component containing the five-membered heterocycle-containing compound with invertebrate pests (including direct consumption of a substance containing the component containing the five-membered heterocycle-containing compound by invertebrate pests, direct contact of a body surface of invertebrate pests with a substance containing the component containing the five-membered heterocycle-containing compound, etc.); indirect way may include treating the place where invertebrate pests infest with substances containing the components of the five-membered heterocyclic compound (their habitat or their breeding ground or the plants, soils on which the invertebrate pests are growing) or treating the food chain with a substance containing a constituent of the five-membered heterocycle-containing compound.

According to a preferred embodiment of the invention, the method comprises: treating a plant on which the invertebrate pest grows with an insecticidal effective amount of at least one of said five-membered heterocycle-containing compounds.

In the present invention, the invertebrate pests are acaridae pests, insects and/or nematodes.

In the present invention, the plant is a plant of phylum gymnospermum and/or angiospermum, and is preferably at least one of a rutaceae plant, a solanaceae plant, a cruciferous plant, and a rosaceae plant.

The present invention does not specifically limit the insecticidal effective amount, but may for example refer to a dosage of 5 g to 3 kg per hectare of said five-membered heterocyclic-containing compounds, which will provide adequate control. Preferably, the insecticidal effective amount may be from 8 grams to 1000 grams per hectare, more preferably from 10 grams to 300 grams per hectare.

It should be clear that various transformations and modifications may be made within the scope limited by the claims of the present invention Compared to the prior art, the present invention has at least the following advantages:

The five-membered heterocyclic-containing compounds provided by the present invention show excellent insecticidal and acaricidal activity against a wide range of pests, in particular against leaf mites represented by the Tetranychus urticae Koch, Tetranychus kanzawai, Panonychus citri Mc Gregor, and other leaf mites, and lepidoptera represented by the Plutella xylostella.

In order to further understand the present invention, the technical solutions in the embodiments of the present invention will be clearly and completely described below with reference to the embodiments of the present invention, and it is obvious that the described embodiments are only a part of the embodiments of the present invention, and not all of the embodiments. All other embodiments, which can be obtained by a person skilled in the art without making any creative effort based on the embodiments in the present invention, belong to the protection scope of the present invention.

The present invention will be described in detail below by way of examples. In the following examples, the various starting materials used are commercially available and are in analytical purity, unless otherwise specified.

### Example 1

### Synthesis of 4-(5-(3, 5-dichloro-4-fluorophenyl)-5-(trifluoromethyl)-4, 5-dihydroisoxazol-3-yl)-N-(1, 1-dioxotetrahydrothiophen-3-yl)-2-methylbenzamide (compound I-1)

### Step 1, synthesizing 4-acetyl-2-methylbenzoic acid

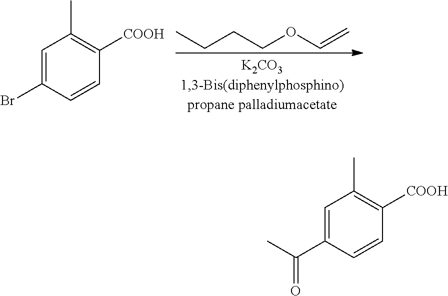

Adding n-butanol (400 mL) into a four-neck flask, then sequentially adding 1, 3-bis (diphenylphosphino) propane (1.15 g), 4-bromo-2-methylbenzoic acid (100 g, 0.465 mol), potassium carbonate (77.12 g, 0.51 mmol) and n-butyl vinyl ether (138.73 g, 1.38 mol), adding palladium acetate (0.21 g) under the protection of nitrogen, and heating and refluxing for reaction for 5 hours under the protection of nitrogen;

After the reaction solution was cooled to 80° C., n-butanol and n-butyl vinyl ether were distilled off under reduced pressure, 500 mL of ethyl acetate and 1000 mL of water were added, liquid separation was performed by extraction after 50 mL of concentrated hydrochloric acid was added, liquid separation was performed by extraction after 500 mL of ethyl acetate was added again to the aqueous phase, the organic phases were combined, dried by adding anhydrous sodium sulfate, and then rotary-evaporated to dryness to obtain 72.5 g of an off-white solid.

### Step 2, synthesizing 1-(3, 5-dichloro-4-fluorophenyl) 2, 2, 2-trifluoroethanone

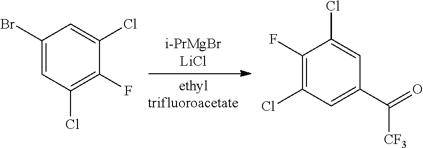 [SMILES: Fc1c(Cl)cc(Br)cc1Cl ; O=C(c1cc(Cl)c(F)c(Cl)c1)C(F)(F)F]

Adding 3, 5-dichloro-4-fluorobromobenzene (122 g, 0.5 mol) into a 3 L four-neck flask, adding tetrahydrofuran (600 mL) under the protection of nitrogen for dissolving, adding lithium chloride (10.5 g, 0.25 mol), dropwise adding 1M isopropyl magnesium bromide (600 mL, 0.6 mol) in an ice bath (below 10° C.), after reacting for 1 h, detecting the reaction degree by a dot plate, dropwise adding ethyl trifluoroacetate (85.2 g, 0.6 mol) after the reaction is finished, and continuously stirring for reacting for 2 h; after the reaction, the reaction solution was added to 2 L of water, 0.2 L of saturated aqueous ammonium chloride solution was added, 1 L ethyl acetate extraction and separation, the aqueous phase was again extracted with 1 L of ethyl acetate, the organic phases were combined, anhydrous sodium sulfate was added, drying was carried out, rotary evaporation was carried out until dry, and distillation under reduced pressure was carried out to obtain 82 g of colorless oil.

### Step 3, synthesizing 4-(3-(3, 5-dichloro-4-fluorophenyl)-4,4, 4-trifluoro-3-hydroxybutyryl)-2-methylbenzoic acid

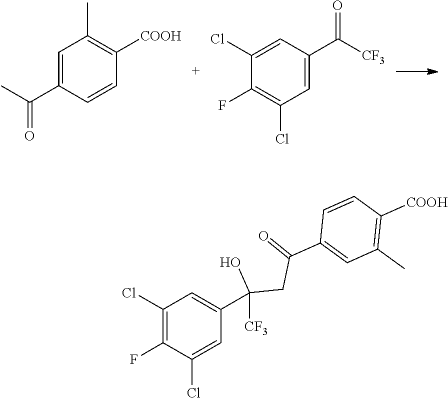 [SMILES: CC(=O)c1ccc(C(=O)O)c(C)c1 ; O=C(c1cc(Cl)c(F)c(Cl)c1)C(F)(F)F ; Cc1cc(C(=O)CC(O)(c2cc(Cl)c(F)c(Cl)c2)C(F)(F)F)ccc1C(=O)O]

Toluene (30 mL) was added to the flask, followed by the sequential addition of 4-acetyl-2-methylbenzoic acid (6.14 g, 34.48 mmol), 1-(3, 5-dichloro-4-fluorophenyl) 2, 2, 2-trifluoroethanone (9.0 g, 34.48 mmol), triethylamine (5.22 g, 51.72 mmol), the reaction flask stirred at 60° C. for 13 hours, and cool the reaction solution to room temperature; the reaction solution was filtered under reduced pressure, and the solid was washed with a small amount of toluene to obtain 13.0 g of a pale yellow solid.

MS: [M−H]⁻:437.15, 439.21.

### Step 4, synthesis of 4-(3-(3, 5-dichloro-4-fluorophenyl)-4,4, 4-trifluoro-2-butenoyl)-2-methylbenzoic acid

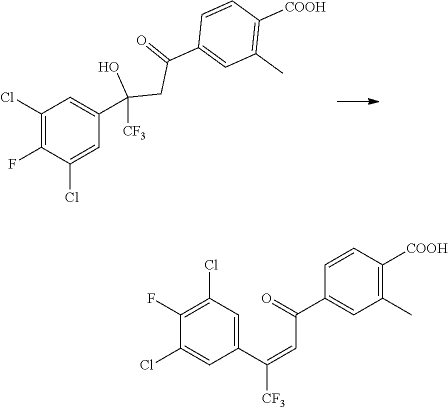 [SMILES: Cc1cc(C(=O)CC(O)(c2cc(Cl)c(F)c(Cl)c2)C(F)(F)F)ccc1C(=O)O ; Cc1cc(C(=O)/C=C(\c2cc(Cl)c(F)c(Cl)c2)C(F)(F)F)ccc1C(=O)O]

4-(3-(3, 5-dichloro-4-fluorophenyl)-4,4, 4-trifluoro-3-hydroxybutyryl)-2-methylbenzoic acid triethylamine salt (10.0 g, 18.49 mmol) was added to a round-bottom flask, a small amount of toluene (30 mL) and 4-dimethylaminopyridine (0.22 g) were added, and the mixture was heated to 60° C. Acetic anhydride (5.66 g, 55.46 mmol) was then added and the mixture was stirred at 60° C. for 9 hours;

The reaction solution was cooled to room temperature, and 20 mL of concentrated hydrochloric acid, 100 mL of water, and 100 mL of ethyl acetate were added. The mixture was extracted and separated, 100 mL of ethyl acetate was added again to the aqueous phase, the organic phases were combined after extraction and separation, and the organic phase was was dried by adding 10 g of anhydrous sodium sulfate and then rotary-evaporated to dryness to obtain 8.5 g of a pale yellow solid compound.

MS: [M−H]⁻:419.21, 421.18.

### Step 5, synthesizing 4-(5-(3, 5-dichloro-4-fluorophenyl)-5-trifluoromethyl-4, 5-dihydroisoxazol-3-yl)-2-methylbenzoic acid

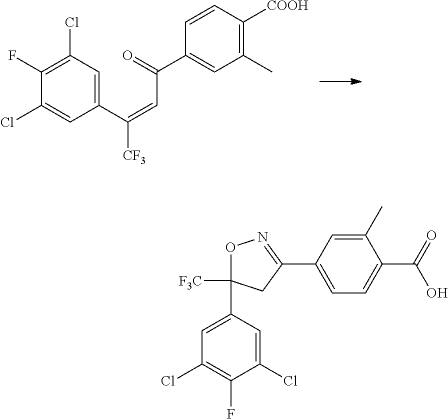 [SMILES: Cc1cc(C(=O)/C=C(\c2cc(Cl)c(F)c(Cl)c2)C(F)(F)F)ccc1C(=O)O ; Cc1cc(C2=NOC(c3cc(Cl)c(F)c(Cl)c3)(C(F)(F)F)C2)ccc1C(=O)O]

Toluene (25 mL) and tetrabutylammonium bromide (0.65 g) were added to a round bottom flask, 4-(3-(3, 5-dichloro-4-fluorophenyl)-4,4, 4-trifluoro-2-butenoyl)-2-methylbenzoic acid (8.5 g, 20 mol) was added, and the reaction flask was stirred at 0 deg.C.; then, sequentially adding sodium hydroxide (3.36 g, 84 mmol), purified water (25 ml) and hydroxylamine hydrochloride (2.78 g, 40 mmol), and continuously stirring the reaction bottle in an ice bath for reaction for 5 hours;

Adding 20 mL of concentrated hydrochloric acid, 100 mL of water and 100 mL of ethyl acetate into the reaction solution; the liquid was separated by extraction, 100 mL of ethyl acetate was added again to the aqueous phase, the organic phases were combined after separation by extraction, and after drying by addition of 10 g of anhydrous sodium sulfate, the organic phase was rotary-evaporated to dryness to obtain 7.2 g of an off-white solid.

MS: [M−H]⁻:434.14, 436.11.

### Step 6, synthesis of 4-(5-(3, 5-dichloro-4-fluorophenyl)-5-(trifluoromethyl)-4, 5-dihydroisoxazol-3-yl)-N-(1, 1-dioxotetrahydrothiophen-3-yl)-2-methylbenzamide (compound I-1)

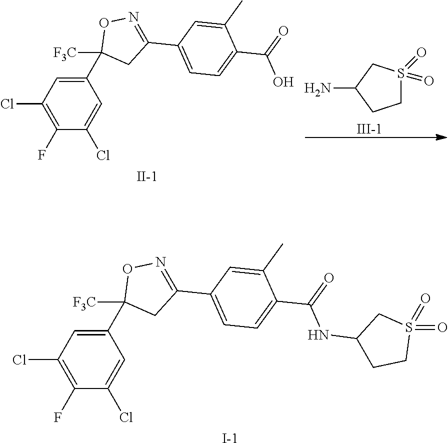

4-(5-(3, 5-dichloro-4-fluorophenyl)-5-trifluoromethyl-4, 5-dihydroisoxazol-3-yl)-2-methylbenzoic acid (4.36 g, 10 mmol) was added to 40 mL of toluene, sulfoxide chloride (1.43 g, 12 mmol) was added, after heating and refluxing for 2 h, depressurize to remove hydrogen chloride and other gases, and dichloroethane (40 mL) was added; and sequentially adding 3-aminotetrahydrothiophene 1, 1-dioxide (2.02 g, 15 mmol) and triethylamine (2.02 g, 20 mmol) into the reaction solution under an ice bath condition, continuously reacting for 1 h, adding 100 mL of water and 50 mL of ethyl acetate into the reaction solution, after extraction and partitioning, the aqueous phase was again added with 50 mL of ethyl acetate, extracted and partitioned again, and the organic phases were combined and evaporated to dryness, and purifying by column chromatography to obtain 4.06 g of a white solid.

¹H NMR (400 MHz, CDCl₃) δ 7.59 (d, J=6.0 Hz, 2H), 7.52 (d, J=8.7 Hz, 2H), 7.43 (d, J=7.9 Hz, 1H), 6.48 (d, J=7.7 Hz, 1H), 4.99 (d, J=6.5 Hz, 1H), 4.08 (d, J=17.2 Hz, 1H), 3.69 (d, J=17.2 Hz, 1H), 3.45 (dd, J=13.8, 7.2 Hz, 1H), 3.33-3.16 (m, 2H), 3.11 (dd, J=14.7, 4.6 Hz, 1H), 2.65 (t, J=7.8 Hz, 1H), 2.48 (s, 3H), 2.42 (d, J=6.3 Hz, 1H).

MS: [M+Na]⁺: 575.43, 577.48.

### Example 2

### Synthesis of 4-(5-(3, 5-dichloro-4-fluorophenyl)-5-(trifluoromethyl)-4, 5-dihydroisoxazol-3-yl)-N—((R)-1, 1-dioxotetrahydrothiophen-3-yl)-2-methylbenzamide (Compound I-14)

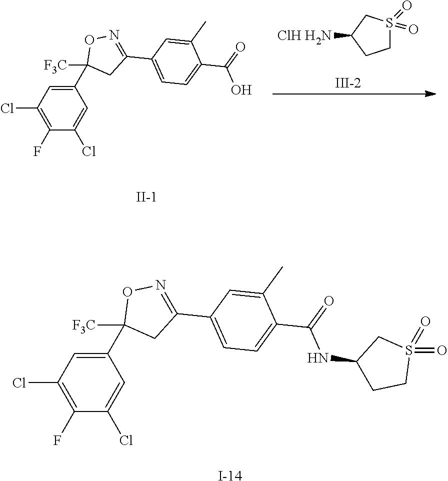

The target compounds were prepared according to the method described in Example 1, with the difference that (R)-3-aminotetrahydrothiophene 1,1-dioxide hydrochloride (Compound III-2) was used instead of 3-aminotetrahydrothiophene 1,1-dioxide (Compound III-1) to prepare to obtain the target compounds I-14, white solid powder.

¹H NMR (400 MHz, Chloroform-d) δ 7.52 (d, J=6.0 Hz, 2H), 7.45 (d, J=9.3 Hz, 2H), 7.35 (d, J=7.9 Hz, 1H), 6.40 (d, J=7.7 Hz, 1H), 4.92 (d, J=7.8 Hz, 1H), 4.01 (d, J=17.2 Hz, 1H), 3.62 (d, J=17.2 Hz, 1H), 3.38 (dd, J=13.8, 7.2 Hz, 1H), 3.25-2.99 (m, 3H), 2.58 (td, J=14.8, 7.3 Hz, 1H), 2.41 (s, 3H), 2.34 (dt, J=14.0, 7.3 Hz, 1H).

MS: [M+Na]⁺: 575.42, 577.38.

### Example 3

### Synthesis of 4-(5-(3, 5-dichloro-4-fluorophenyl)-5-(trifluoromethyl)-4, 5-dihydroisoxazol-3-yl)-N—((S)-1, 1-dioxotetrahydrothiophen-3-yl)-2-methylbenzamide (Compound I-13)

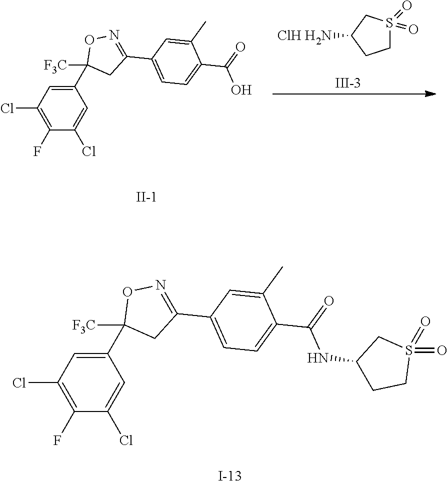

The target compounds were prepared according to the method described in Example 1, with the difference that (S)-3-aminotetrahydrothiophene 1,1-dioxide hydrochloride (Compound III-3) was used instead of 3-aminotetrahydrothiophene 1,1-dioxide (Compound III-1) to prepare the target compounds I-13, white solid powder.

¹H NMR (400 MHz, Chloroform-d) δ 7.59 (d, J=6.0 Hz, 2H), 7.51 (d, J=9.4 Hz, 2H), 7.42 (d, J=7.9 Hz, 1H), 6.49 (d, J=7.6 Hz, 1H), 4.98 (d, J=6.6 Hz, 1H), 4.16-4.08 (m, 1H), 3.69 (d, J=17.2 Hz, 1H), 3.44 (dd, J=13.8, 7.2 Hz, 1H), 3.32-3.05 (m, 3H), 2.65 (ddt, J=14.7, 9.0, 4.4 Hz, 1H), 2.47 (s, 3H), 2.40 (dt, J=13.9, 7.0 Hz, 1H).

MS: [M+Na]⁺: 575.38, 577.36.

### Example 4

### Synthesis of 4-(5-(3, 5-dichloro-4-fluorophenyl)-5-(trifluoromethyl)-4, 5-dihydroisoxazol-3-yl)-2-methyl-N-(tetrahydrothiophen-3-yl) benzamide (compound I-21)

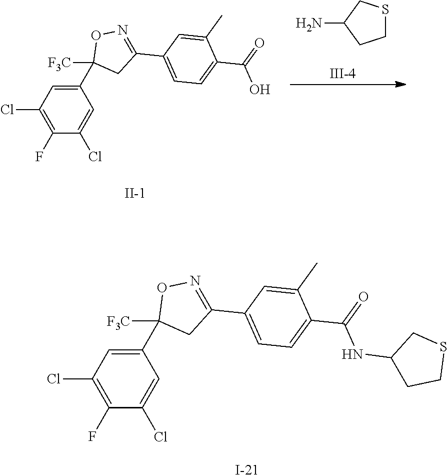

The target compound was prepared according to the method described in Example 1, with the difference that 3-amino tetrahydrothiophene (Compound III-4) was used instead of 3-amino tetrahydrothiophene 1,1-dioxide (Compound III-1), to obtain the target compound I-21, a white solid powder.

MS: [M+H]⁺: 521.33; [M+Na]⁺: 543.32.

### Example 5

### Synthesis of N-(1, 1-dioxotetrahydrothiophen-3-yl)-2-methyl-4-(5-(3,4, 5-trichlorophenyl)-5-(trifluoromethyl)-4, 5-dihydroisoxazol-3-yl) benzamide (compound I-3)

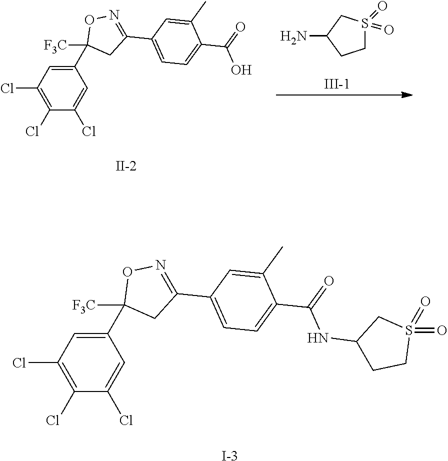

The target compound was prepared according to the method described in Example 1, with the difference that (4-(5-(3,4,5-trichlorophenyl)-5-trifluoromethyl-4,5-dihydroisoxazol-3-yl)-2-methylbenzoic acid (Compound II-2) was used instead of 4-(5-(3,5-dichloro-4-fluorophenyl)-5-trifluoromethyl-4,5-dihydroisoxazol-3-yl)-2-methylbenzoic acid (Compound II-1) was prepared to obtain the target compound I-13, white solid powder.

¹H NMR (400 MHz, CDCl₃) δ 7.65 (s, 2H), 7.57-7.48 (m, 2H), 7.42 (d, J=8.0 Hz, 1H), 6.44 (d, J=7.6 Hz, 1H), 4.99 (d, J=5.9 Hz, 1H), 4.09 (d, J=17.2 Hz, 1H), 3.69 (d, J=17.3 Hz, 1H), 3.45 (dd, J=13.8, 7.2 Hz, 1H), 3.35-3.15 (m, 2H), 3.11 (dd, J=13.8, 3.8 Hz, 1H), 2.74-2.59 (m, 1H), 2.47 (s, 3H), 2.43 (s, 1H).

MS: [M+H]⁺: 569.03, 571.27, 573.32.

### Example 6

### Synthesis of methyl 2-(4-(5-(3, 5-dichloro-4-fluorophenyl)-5-(trifluoromethyl)-4, 5-dihydroisoxazol-3-yl)-N-(1, 1-dioxotetrahydrothiophen-3-yl)-2-methylbenzamido)-2-methyl oxyacetate (compound I-12)

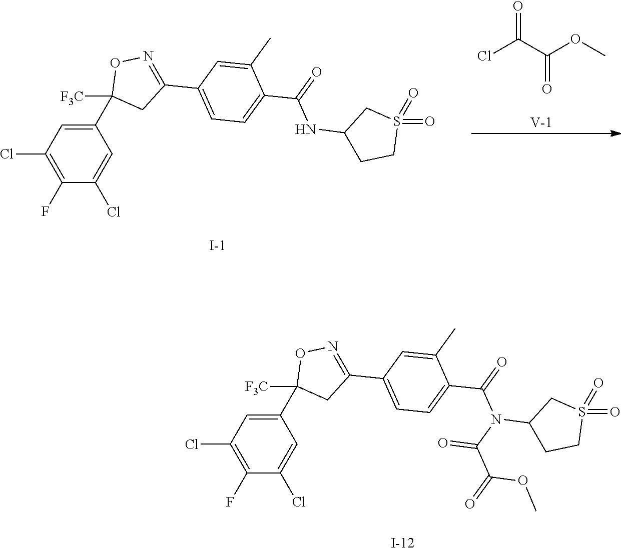

Adding 4-(5-(3, 5-dichloro-4-fluorophenyl)-5-(trifluoromethyl)-4, 5-dihydroisoxazol-3-yl)-N-(1, 1-dioxotetrahydrothiophen-3-yl)-2-methylbenzamide (553 mg, 1 mmol) into a round-bottom flask, adding dichloroethane (10 mL), then sequentially adding triethylamine (303 mg, 3 mmol) and oxalyl chloride monomethyl ester (245 mg, 2 mmol), heating the reaction solution to reflux, cooling the reaction solution to room temperature after 3 hours, adding 50 mL of water and 50 mL of ethyl acetate, washing the organic phase after liquid separation by adding saturated common salt water, drying by anhydrous sodium sulfate, concentrating the organic phase to dryness under pressure, and purifying by column chromatography to obtain a white solid 303 mg.

MS: [M+Na]⁺: 661.39, 663.37.

### Example 7

### Synthesis of N-acetyl-4-(5-(3, 5-dichloro-4-fluorophenyl)-5-(trifluoromethyl)-4, 5-dihydroisoxazol-3-yl)-N-(1, 1-dioxotetrahydrothiophen-3-yl)-2-methylbenzamide (compound I-9)

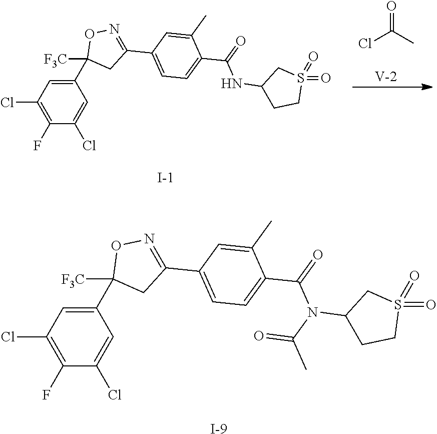

The target compounds were prepared according to the method described in Example 6, with the difference that acetyl chloride (Compound V-2) was used instead of oxalyl chloride monomethyl ester (Compound V-1) to prepare the target compounds I-9, a white solid powder.

¹H NMR (400 MHz, CDCl₃) δ 7.66-7.54 (m, 4H), 7.31 (d, J=8.0 Hz, 1H), 4.98-4.84 (m, 1H), 4.10 (d, J=17.2 Hz, 1H), 3.70 (d, J=17.2 Hz, 3H), 3.22-2.97 (m, 2H), 2.63-2.49 (m, 2H), 2.47 (s, 3H), 2.12 (s, 3H).

MS: [M+H]⁺: 595.33; [M+Na]⁺: 617.32.

### Example 8

### Synthesis of N-propionyl-4-(5-(3, 5-dichloro-4-fluorophenyl)-5-(trifluoromethyl)-4, 5-dihydroisoxazol-3-yl)-N-(1, 1-dioxotetrahydrothiophen-3-yl)-2-methylbenzamide (compound I-10)

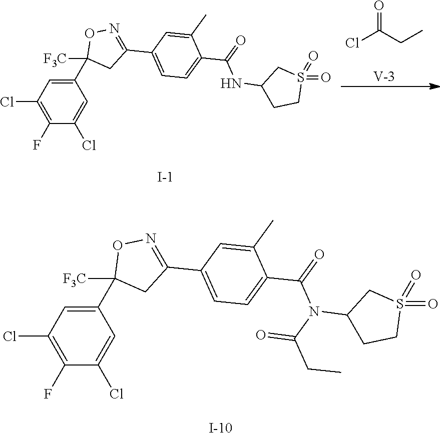

The target compound was prepared according to the method described in Example 6, with the difference that propionyl chloride (Compound V-3) was used instead of oxalyl chloride monomethyl ester (Compound V-1) to prepare the target compound I-10, a white solid powder.

¹H NMR (400 MHz, CDCl₃) δ 7.59 (dd, J=13.3, 7.4 Hz, 4H), 7.29 (d, J=8.0 Hz, 1H), 4.90 (s, 1H), 4.10 (d, J=17.2 Hz, 1H), 3.77-3.52 (m, 3H), 3.22-3.03 (m, 2H), 2.56-2.43 (m, 5H), 2.36 (dd, J=14.6, 7.3 Hz, 2H), 1.05 (t, J=7.3 Hz, 3H).

MS: [M+Na]⁺: 631.32, 633.29.

### Example 9

### Synthesis of N-isopropionyl-4-(5-(3, 5-dichloro-4-fluorophenyl)-5-(trifluoromethyl)-4, 5-dihydroisoxazol-3-yl)-N-(1, 1-dioxotetrahydrothiophen-3-yl)-2-methylbenzamide (compound I-21)

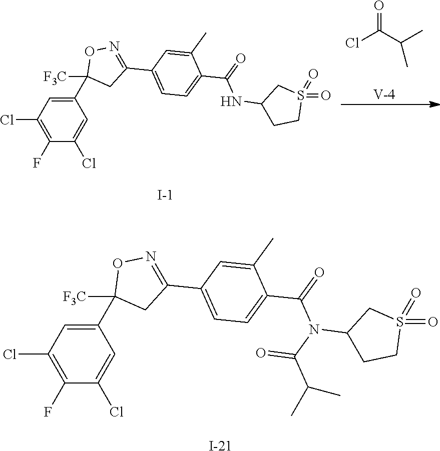

The target compound was prepared according to the method described in Example 6, with the difference that isobutyryl chloride (Compound V-4) was used instead of oxalyl chloride monomethyl ester (Compound V-1) to prepare the target compound I-21, a white solid powder.

¹H NMR (400 MHz, CDCl₃) δ 7.59 (dd, J=15.1, 9.1 Hz, 4H), 7.28 (s, 1H), 5.07-4.91 (m, 1H), 4.10 (d, J=17.2 Hz, 1H), 3.76-3.54 (m, 3H), 3.17 (dd, J=12.6, 8.6 Hz, 1H), 3.14-3.02 (m, 1H), 2.63 (dd, J=13.3, 6.7 Hz, 1H), 2.56-2.45 (m, 5H), 1.00 (dt, J=14.8, 7.4 Hz, 6H).

MS: [M+H]⁺: 623.26; [M+Na]⁺: 645.28.

### Example 10

### Synthesis of N-cyclopropanoyl-4-(5-(3, 5-dichloro-4-fluorophenyl)-5-(trifluoromethyl)-4, 5-dihydroisoxazol-3-yl)-N-(1, 1-dioxotetrahydrothiophen-3-yl)-2-methylbenzamide (compound I-22)

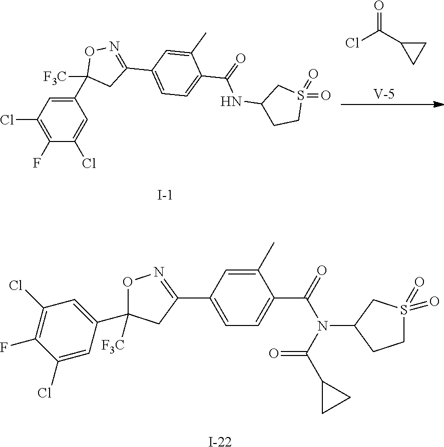

The target compound was prepared according to the method described in Example 6, with the difference that cyclopropylcarbonyl chloride (Compound V-5) was used instead of oxalyl chloride monomethyl ester (Compound V-1) to prepare the target compound I-22, a white solid powder.

¹H NMR (400 MHz, CDCl₃) δ 7.65-7.51 (m, 4H), 7.34 (d, J=8.0 Hz, 1H), 5.29 (dt, J=16.7, 8.4 Hz, 1H), 4.09 (d, J=17.2 Hz, 1H), 3.69 (d, J=17.4 Hz, 2H), 3.56 (dt, J=12.5, 8.0 Hz, 1H), 3.26 (dd, J=12.5, 8.4 Hz, 1H), 3.21-3.03 (m, 1H), 2.57 (s, 2H), 2.49 (s, 3H), 1.55 (s, 3H), 1.16-0.98 (m, 2H).

MS: [M+Na]⁺: 643.36.

### Example 11

### Synthesis of 4-(5-(3, 5-dichloro-4-fluorophenyl)-5-(trifluoromethyl)-4, 5-dihydroisoxazol-3-yl)-N-(1, 1-dioxotetrahydrothiophen-3-yl)-N, 2-dimethylbenzamide (compound I-7)

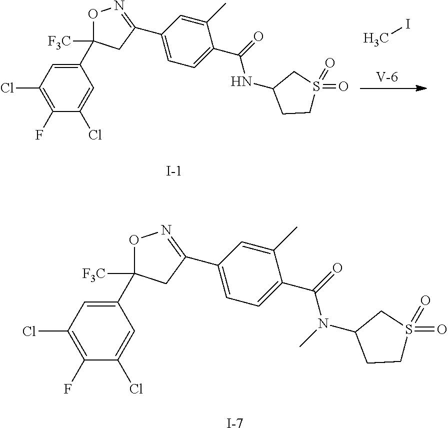

Adding 4-(5-(3, 5-dichloro-4-fluorophenyl)-5-(trifluoromethyl)-4, 5-dihydroisoxazol-3-yl)-N-(1, 1-dioxy tetrahydrothiophene-3-yl)-2-methylbenzamide (553 mg, 1 mmol) into a pressure-resistant bottle, adding anhydrous tetrahydrofuran (5 mL), cooling to 0° C. in an ice bath, iodomethane (213 mg, 15 mmol) was then added with a syringe, reaction bottles are sealed and warmed to 100° C., heating and stirring for 3 hours, cooling to room temperature, adding 50 mL of water and 50 mL of ethyl acetate, adding saturated salt into an organic phase after extraction and liquid separation, washing, after drying with anhydrous sodium sulfate, the organic phase is concentrated to dryness under reduced pressure, and purifying by column chromatography to obtain a white solid of 110 mg.

¹H NMR (400 MHz, CDCl₃) δ 7.59 (d, J=6.0 Hz, 4H), 7.22 (d, J=7.8 Hz, 1H), 4.08 (d, J=17.1 Hz, 1H), 3.70 (dd, J=16.8, 11.0 Hz, 1H), 3.56-3.34 (m, 2H), 3.14 (dd, J=23.1, 14.5 Hz, 3H), 2.79 (s, 3H), 2.57-2.34 (m, 2H), 2.30 (s, 3H).

MS: [M+H]⁺: 567.28; [M+Na]⁺: 589.18.

### Example 12

### Synthesis of 4-(5-(3, 5-bis(trifluoromethyl) phenyl)-5-(trifluoromethyl ester)-4, 5-dihydroisoxazol-3-yl)-N-(1, 1-dioxotetrahydrothiophen-3-yl)-2-methylbenzamide (Compound I-25)

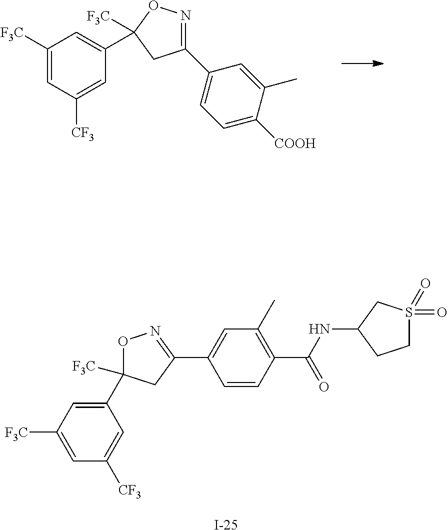

4-(5-(3, 5-bis(trifluoromethyl) phenyl)-5-(trifluoromethyl ester)-4, 5-dihydroisoxazol-3-yl)-2-methylbenzoic acid (485 mg, 1 mmol) is added to dichloroethane, diisopropylethylamine (258 mg, 2 mmol) and 2-(7-azabenzotriazole)-N, N, N′, N′-tetramethyluronium hexafluorophosphate (494 mg, 1.3 mmol) are added, after stirring for 30 minutes, 3-aminotetrahydrothiophene 1, 1-dioxide (202.5 mg, 1.5 mmol) is added, continue to stir the reaction for 30 minutes, the reaction solution is added with 50 mL of water and 50 mL of dichloromethane, after extraction and liquid separation, the organic phase is concentrated to dryness under reduced pressure, and column chromatography purification is carried out to obtain 426 mg of white solid.

¹H NMR (400 MHz, CDCl₃) δ 8.08 (s, 2H), 7.97 (s, 1H), 7.53 (d, J=8.4 Hz, 2H), 7.42 (d, J=8.0 Hz, 1H), 6.53 (d, J=7.6 Hz, 1H), 4.97 (dd, J=10.9, 5.5 Hz, 1H), 4.20 (d, J=17.3 Hz, 1H), 3.76 (d, J=17.3 Hz, 1H), 3.44 (dd, J=13.8, 7.2 Hz, 1H), 3.32-3.14 (m, 2H), 3.14-3.03 (m, 1H), 2.73-2.54 (m, 1H), 2.47 (s, 3H), 2.46-2.30 (m, 1H).

MS: [M+Na]⁺: 625.24.

### Example 13

### Step 1: Preparation of methyl (S)-4-(3-(3, 5-dichloro-4-fluorophenyl)-4, 4-4-trifluoro-3-hydroxybutyryl)-2-methylbenzoate

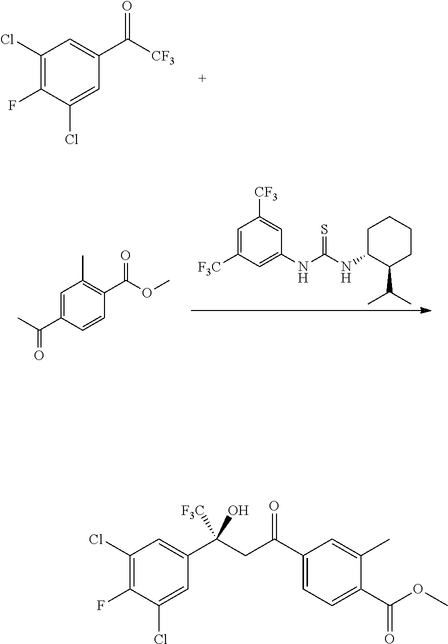 [SMILES: O=C(c1cc(Cl)c(F)c(Cl)c1)C(F)(F)F ; COC(=O)c1ccc(C(C)=O)cc1C ; CC(C)[C@@H]1CCCC[C@H]1NC(=S)Nc1cc(C(F)(F)F)cc(C(F)(F)F)c1 ; COC(=O)c1ccc(C(=O)C[C@](O)(c2cc(Cl)c(F)c(Cl)c2)C(F)(F)F)cc1C]

To a solution of 1-(3, 5-dichloro-4-fluorophenyl)-2,2, 2-trifluoro-ethanone (10.44 g, 40 mmol) in toluene (42 mL) were added 1-[3, 5-bis(trifluoromethyl) phenyl]-3-[(1R,2R)-(−)-2-(dimethylamino) cyclohexyl]thiourea (1.5 g, 3.65 mmol) and 4-acetyl-2-methyl-benzoic acid methyl ester (7.0 g, 36.5 mmol) and the mixture was stirred at 20 to 25° C. overnight. After the reaction was completed, the solvent was removed by concentration under reduced pressure, and the residue was purified by column chromatography to obtain 4.5 g of a white solid, which was the title compound.

¹H NMR (400 MHz, Chloroform-d) δ 8.00 (d, J=8.6 Hz, 1H), 7.81-7.74 (m, 2H), 7.56 (d, J=6.1 Hz, 2H), 5.66 (s, 1H), 3.94 (s, 3H), 3.84 (d, J=17.6 Hz, 1H), 3.69 (d, J=17.6 Hz, 1H), 2.66 (s, 3H).

MS: [M+Na]⁺: 475.12, 477.36.

### Step 2: Preparation of methyl (S, Z)-4-(3-(3, 5-dichloro-4-fluorophenyl)-4, 4-4-trifluoro-3-hydroxy-1-(hydroxyimino) butyl)-2-methylbenzoate

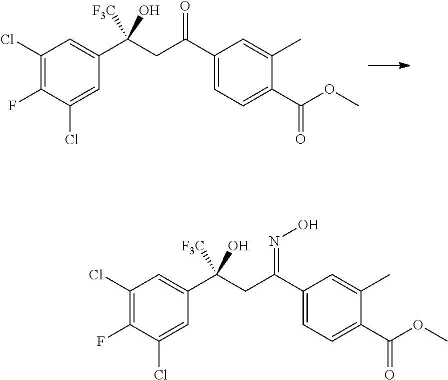 [SMILES: COC(=O)c1ccc(C(=O)C[C@](O)(c2cc(Cl)c(F)c(Cl)c2)C(F)(F)F)cc1C ; COC(=O)c1ccc(/C(C[C@](O)(c2cc(Cl)c(F)c(Cl)c2)C(F)(F)F)=N\O)cc1C]

To a mixture of methyl (S)-4-(3-(3, 5-dichloro-4-fluorophenyl)-4, 4-4-trifluoro-3-hydroxybutyryl)-2-methylbenzoate (1 g, 2.2 mmol) and 2, 6-dimethylpyridine (7 ml) was added solid hydroxylamine hydrochloride (0.31 g, 4.4 mmol) at 20 to 25° C. After stirring overnight, the system was quenched by the addition of 10 ml of ice water and the layers were extracted with dichloromethane and the combined organic layers were washed with aqueous hydrochloric acid (6N) and water, dried over anhydrous sodium sulfate, filtered, concentrated in vacuo. 0.6 g of a light brown oil is obtained, which is the title compound.

¹H NMR (400 MHz, Chloroform-d) δ 7.89 (d, J=8.1 Hz, 1H), 7.41 (d, J=6.1 Hz, 2H), 7.06 (dd, J=8.2, 1.8 Hz, 1H), 6.99 (d, J=1.8 Hz, 1H), 5.23 (s, 1H), 3.91 (s, 3H), 3.32 (d, J=1.1 Hz, 2H), 2.56 (s, 3H).

MS: [M+Na]⁺:490.49, 492.46.

### Step 3: Preparation of methyl (S)-4-(5-(3, 5-dichloro-4-fluorophenyl)-5-(trifluoromethyl)-4, 5-dihydroisoxazol-3-yl)-2-methylbenzoate

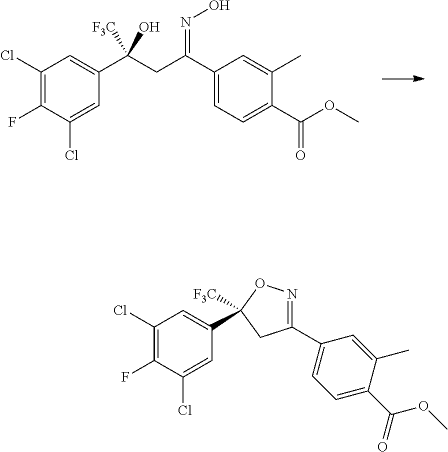 [SMILES: COC(=O)c1ccc(/C(C[C@](O)(c2cc(Cl)c(F)c(Cl)c2)C(F)(F)F)=N\O)cc1C ; COC(=O)c1ccc(C2=NO[C@@](c3cc(Cl)c(F)c(Cl)c3)(C(F)(F)F)C2)cc1C]

To a solution of methyl (S, Z)-4-(3-(3, 5-dichloro-4-fluorophenyl)-4, 4-4-trifluoro-3-hydroxy-1-(hydroxyimino) butyl)-2-methylbenzoate (1 g, 2.14 mmol) in anhydrous tetrahydrofuran (10 ml) was added triphenylphosphine (0.84 g, 3.21 mmol), diethyl azodicarboxylate (0.56 g, 3.21 mmol) was added in portions below 20° C., and the mixture was stirred at room temperature overnight. After the reaction was complete, 20 ml of water were added to the system and the fractions were extracted with dichloromethane, dried over anhydrous sodium sulfate, filtered and concentrated in vacuo, and the concentrate was purified by rapid chromatography on silica gel to give 0.5 g of colorless oil, which was the title compound.

¹H NMR (400 MHz, CDCl₃) δ 8.08-7.92 (m, 1H), 7.59 (d, J=6.0 Hz, 2H), 7.53 (d, J=4.5 Hz, 2H), 4.10 (d, J=17.2 Hz, 1H), 3.91 (s, 3H), 3.70 (d, J=17.2 Hz, 1H), 2.63 (s, 3H).

MS: [M+H]⁺:450.50, 452.54.

### Step 4: Preparation of (S)-4-(5-(3, 5-dichloro-4-fluorophenyl)-5-(trifluoromethyl)-4, 5-dihydroisoxazol-3-yl)-2-methylbenzoic acid

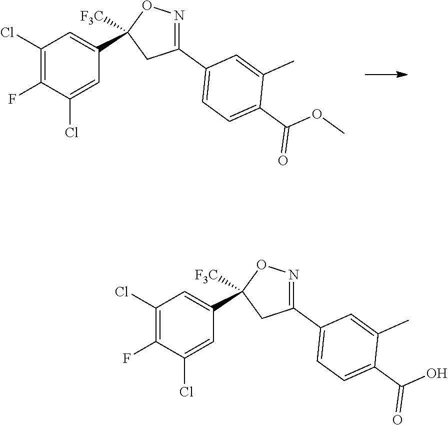 [SMILES: COC(=O)c1ccc(C2=NO[C@@](c3cc(Cl)c(F)c(Cl)c3)(C(F)(F)F)C2)cc1C ; Cc1cc(C2=NO[C@@](c3cc(Cl)c(F)c(Cl)c3)(C(F)(F)F)C2)ccc1C(=O)O]

To a solution of (S)-methyl 4-(5-(3, 5-dichloro-4-fluorophenyl)-5-(trifluoromethyl)-4, 5-dihydroisoxazol-3-yl)-2-methylbenzoate (0.5 g, 1.11 mmol) in tetrahydrofuran (3 ml) was added 10% aqueous lithium hydroxide (0.5 g), the reaction was warmed to 70° C. and stirred for 5 hours to complete the reaction, the system was cooled to room temperature and adjusted to pH 2 to 3 with 6M aqueous hydrochloric acid, and after extraction with dichloromethane, the mixture was dried and desolventized to give 0.3 g of a light brown oil.

MS: [M+H]⁻: 434.57, 436.56.

### Step 5: Preparation of 4-((S)-5-(3, 5-dichloro-4-fluorophenyl)-5-(trifluoromethyl)-4, 5-dihydroisoxazol-3-yl)-N-(1, 1-dioxotetrahydrothiophen-3-yl)-2-methylbenzamide (Compound I-17)

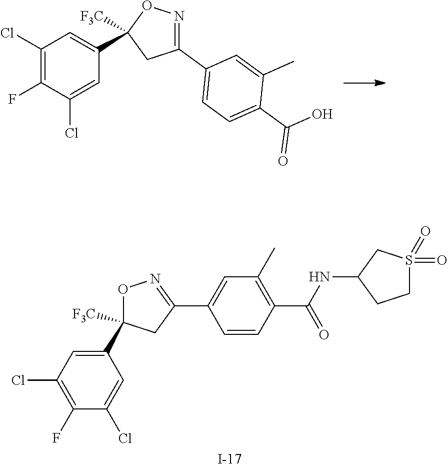

To a solution of (S)-4-(5-(3, 5-dichloro-4-fluorophenyl)-5-(trifluoromethyl)-4, 5-dihydroisoxazol-3-yl)-2-methylbenzoic acid (0.3 g, 0.688 mmol) in dichloromethane (5 ml) were added 2-(7-azabenzotriazole)-N, N, N′, N′-tetramethyluronium hexafluorophosphate (0.39 g, 1.03 mmol) and N, N-diisopropylethylamine (0.18 g, 1.38 mmol), and after stirring at room temperature for 0.5, 3-Aminotetrahydrothiophene1,1-dioxidehydrochloride (0.18 g, 1.03 mmol) was added to the system, and the mixture was stirred at room temperature overnight. After the reaction was complete, 10 ml of water were added to the system and the fractions were extracted with dichloromethane, dried over anhydrous sodium sulfate, filtered and concentrated in vacuo, and the concentrate was purified by rapid chromatography on silica gel to give 0.28 g of colorless oil, which was the title compound.

¹H NMR (400 MHz, CDCl₃) δ 7.59 (d, J=6.0 Hz, 2H), 7.51 (d, J=9.4 Hz, 2H), 7.41 (d, J=7.9 Hz, 1H), 6.52 (d, J=7.7 Hz, 1H), 4.97 (dd, J=10.9, 5.7 Hz, 1H), 4.17 (d, J=17.2 Hz, 1H), 3.70 (d, J=17.2 Hz, 1H), 3.44 (dd, J=13.8, 7.2 Hz, 1H), 3.35-3.14 (m, 2H), 3.10 (dd, J=13.8, 4.1 Hz, 1H), 2.65 (ddd, J=21.8, 8.7, 6.6 Hz, 1H), 2.48 (d, J=11.6 Hz, 3H) 2.40 (dd, J=13.9, 6.9 Hz, 1H).

MS: [M+H]⁺:553.46, 555.47.

### Comparative Example 1

### Synthesis of 4-(5-(3, 5-dichloro-4-fluorophenyl)-5-(trifluoromethyl)-4, 5-dihydroisoxazol-3-yl)-N-(1, 1-dioxothiophen-3-yl)-2-methylbenzamide (compound x.82 in CN 106045962A)

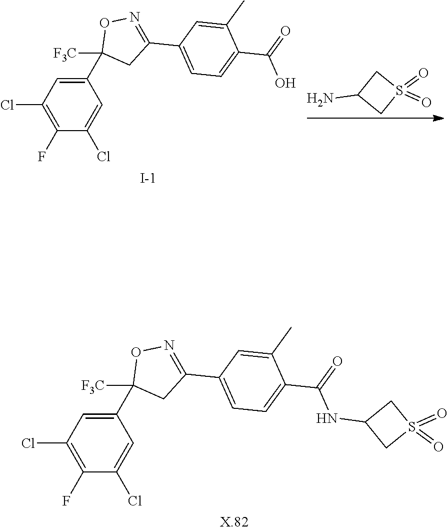 [SMILES: Cc1cc(C2=NOC(c3cc(Cl)c(F)c(Cl)c3)(C(F)(F)F)C2)ccc1C(=O)O ; NC1CS(=O)(=O)C1 ; [I-] ; Cc1cc(C2=NOC(c3cc(Cl)c(F)c(Cl)c3)(C(F)(F)F)C2)ccc1C(=O)NC1CS(=O)(=O)C1 ; "X.82"]

The objective compound X.82 was obtained as a white solid powder by the method described in example 1, using 3-aminothietane 1,1-dioxide (cas number: 88511-13-1) in place of 3-aminotetrahydrothiophene 1, 1-dioxide (compound III-1).

¹H NMR (400 MHz, CDCl₃) δ 7.59 (d, J=6.0 Hz, 2H), 7.54 (d, J=7.3 Hz, 2H), 7.46 (s, 1H), 6.50 (d, J=6.8 Hz, 1H), 4.89 (dd, J=9.4, 6.0 Hz, 1H), 4.62 (s, 2H), 4.13-3.99 (m, 3H), 3.71 (s, 1H), 2.48 (s, 3H).

MS: [M+H]⁺: 539.15, 541.12; [M+Na]⁺: 561.19, 563.14.

### Comparative Example 2

### Synthesis of 4-[5-(3, 5-dichlorophenyl)-4, 5-dihydro-5-(trifluoromethyl)-3-isoxazolyl]-N-[(methoxyamino) methylene]-2-methylbenzamide (fluorooxazolamide)

### Step 1, synthesis of 4-(5-(3, 5-dichlorophenyl)-5-(trifluoromethyl)-4, 5-dihydroisoxazol-3-yl)-2-methylbenzamide

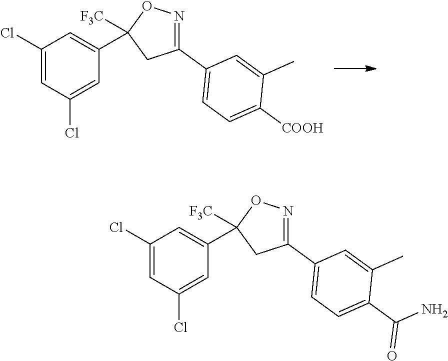 [SMILES: Cc1cc(C2=NOC(c3cc(Cl)cc(Cl)c3)(C(F)(F)F)C2)ccc1C(=O)O ; Cc1cc(C2=NOC(c3cc(Cl)cc(Cl)c3)(C(F)(F)F)C2)ccc1C(N)=O]

4-(5-(3, 5-dichloro-phenyl)-5-trifluoromethyl-4, 5-dihydroisoxazol-3-yl)-2-methylbenzoic acid (4.18 g, 10 mmol) was added to 40 mL of toluene, sulfoxide chloride (1.43 g, 12 mmol) was added, after heating and refluxing for 2 h, depressurized to remove hydrogen chloride and other gases, and tetrahydrofuran (40 mL) was added; adding ammonia water (10 mL) into the reaction liquid under an ice bath condition, continuously reacting for 1 h, adding 100 mL of water and 50 mL of ethyl acetate into the reaction liquid, adding 50 mL of ethyl acetate into the water phase after extraction and liquid separation, the liquid was extracted again, and the organic phases were combined and evaporated to dryness, performing rotary evaporation till dryness, and purifying by column chromatography to obtain 3.6 g of white solid.

MS: [M+H]⁺: 417.07, 418.99.

### Step 2, Synthesis of 4-[5-(3, 5-dichlorophenyl)-4, 5-dihydro-5-(trifluoromethyl)-3-isoxazolyl]-N-[(methoxyamino)methylene]-2-methylbenzamide (fluorooxazoleamide)

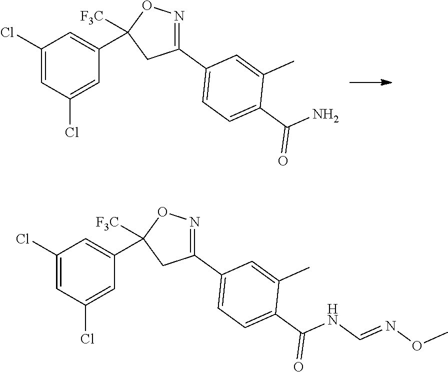 [SMILES: Cc1cc(C2=NOC(c3cc(Cl)cc(Cl)c3)(C(F)(F)F)C2)ccc1C(N)=O ; CO/N=C/NC(=O)c1ccc(C2=NOC(c3cc(Cl)cc(Cl)c3)(C(F)(F)F)C2)cc1C]

4-(5-(3, 5-dichlorophenyl)-5-(trifluoromethyl)-4, 5-dihydroisoxazol-3-yl)-2-methylbenzamide (1.5 g, 3.6 mmol) was added to N, N-dimethylformamide dimethyl acetal (10 mL), heated at 110° C. under reflux for 3 hours and then dioxane (5 mL) was added.

In a round-bottomed flask, methoxyamine hydrochloride (0.60 g, 7.2 mmol) was added, an aqueous solution (8 mL) of sodium hydroxide (0.56 g, 14.4 mmol) was added, 16 mL of glacial acetic acid was added, and after stirring for 5 minutes, the above dioxane solution was slowly added dropwise. After stirring at room temperature for 30 minutes, 50 mL of ethyl acetate and 50 mL of water were added, and after extraction, the organic phase was dried over anhydrous sodium sulfate, concentrated to dryness under reduced pressure, and the concentrate was purified by column chromatography to give 0.64 g of a white solid.

¹H NMR (400 MHz, CDCl₃) δ 8.53 (d, J=9.7 Hz, 1H), 7.75 (d, J=9.3 Hz, 1H), 7.61-7.47 (m, 5H), 7.43 (t, J=1.8 Hz, 1H), 4.10 (d, J=17.3 Hz, 1H), 3.90 (d, J=6.3 Hz, 3H), 3.76-3.63 (m, 1H), 2.52 (s, 3H).

MS: [M+H]⁺: 474.13, 476.07.

### Application Example 1

Tetranychus cinnabarinus Controlling Activity Test:

The test compound was dissolved in acetone and diluted to the desired concentration with 0.1 wt % aqueous tween 80, the acetone content not exceeding 5 wt %.

One real leaf was removed from kidney bean seedlings grown to two real leaves, inoculating Tetranychus cinnabarinus, investigating the base number, performing whole plant spraying treatment by using a handheld sprayer, repeating the treatment for 3 times (the application amount of the preparation is about 0.5 g), observing in a constant-temperature observation room after the treatment, investigating the number of live mites after 72 hours, and calculating the mortality. The number of Tetranychus cinnabarinus inoculated in each time is 35-100.

Mortality%=(number of inoculated insects−number of live insects after drug administration)÷number of inoculated insects×100%

In this test, Compound I-1, Compound I-3, Compound I-9, Compound I-10, Compound I-13, Compound I-14, Compound I-15, Compound I-16, Compound I-17, Compound I-18, Compound I-21, Compound I-22, Compound I-23, Compound I-25 each showed a mortality of more than 9000 against Tetranychus cinnabarinus at 3.12 pppm (3.125 mg/L).

In this test, Compound I-1, Compound I-3, Compound I-9, Compound I-10, Compound I-14, Compound I-17, Compound I-25 each showed a mortality of more than 90 against Tetranychus cinnabarinus at 1.6 ppm (1.56 mg/L).

According to the method, the compound I-1, the compound I-10, the compound I-14, the compound I-25, the compound I-17 and the fluxapyroxad are selected to be respectively subjected to an acaricidal (Tetranychus cinnabarinus) parallel test. The results of the tests are given in table 1 below.

TABLE 1

| | | Mortality (%) | | | |
|---|---|---|---|---|---|
| Compound | | 12.5 | 6.25 | 3.125 | 1.56 |
| number | Structural formula | ppm | ppm | ppm | ppm |
| Compounds I-1 of the invention | 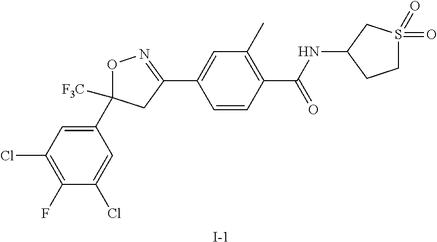 [SMILES: Cc1cc(C2=NOC(c3cc(Cl)c(F)c(Cl)c3)(C(F)(F)F)C2)ccc1C(=O)NC1CCS(=O)(=O)C1 ; [I-]] | 100 | 100 | 100 | 94.32 |
| Compounds I-10 of the invention | 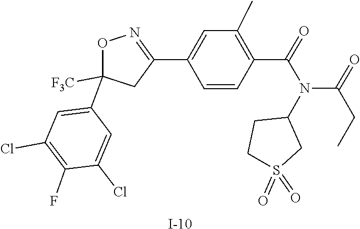 | 100 | 100 | 100 | 97.16 |
| Compounds I-14 of the invention | 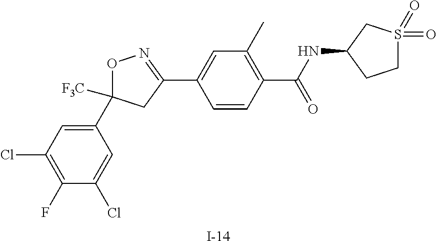 | 100 | 100 | 100 | 100 |
| Compounds I-25 of the invention | 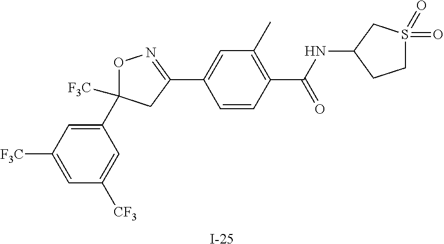 | 100 | 100 | 99.32 | 90.29 |
| Compounds I-17 of the invention | 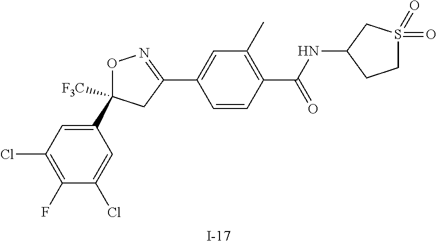 | 100 | 100 | 100 | 100 |
| Fluxametamide | 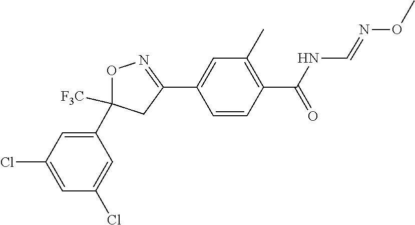 [SMILES: CO/N=C/NC(=O)c1ccc(C2=NOC(c3cc(Cl)cc(Cl)c3)(C(F)(F)F)C2)cc1C] | 96.83 | 81.48 | 25.57 | 0 |

| | | |
|---|---|---|
| Blank control | — | 2.73 |

As can be seen from the above, the five-membered heterocyclic-containing compounds of the present invention, when used at lower concentrations, still have a high acaricidal effect on Tetranychus cinnabarinus, and the acaricidal effect is significantly better than that of the marketed insecticide and acaricide, fluxametamide.

### Application Example 2

Spodoptera frugiperda Indoor Toxicity Determination Test

Test targets: Spodoptera frugiperda, 3rd instar larvae, a sensitive strain raised indoors.

The test method comprises the following steps: firstly, selecting fresh corn leaves cultivated in a greenhouse, cutting the fresh corn leaves into 5 cm-long leaf segments, sequentially arranging the test design from low dose to high dose, soaking the fresh corn leaves in prepared liquid medicine for 10 s, naturally drying the fresh corn leaves in the shade, then placing the dried corn leaves in a culture dish with filter paper and the diameter of 9 cm, inoculating regular healthy test insects, n=10, repeating the treatment for 3 times, and additionally arranging blank control.

Compound I-1, compound I-10, compound I-14, compound I-25 showed more than 90% mortality against Spodoptera frugiperda at 1 ppm (1 mg/L).

### Application Example 3

Phyllotreta striolata Controlling Activity Test:

Preparing liquid medicine according to the experimental design concentration, selecting fresh cabbage leaves, soaking the cabbage leaves for 15 s, taking out, airing, placing in a glass tube, connecting 20-25 heads of the Flea beetles adults with each tube, repeating the treatment for 3 times, investigating the death condition of the Phyllotreta striolata after 72 hours, and calculating and correcting the death rate.

In this test, Compound I-1, Compound I-14, Compound I-17, and Compound I-25 each showed a mortality of more than 90% at 50 ppm (50 mg/L) against Phyllotreta striolata.

### Application Example 4

Frankliniella occidentalis Larva Controlling Activity Test:

The test compound is dissolved in acetone and diluted to the desired concentration. 0.5 mL of the prepared acetone medicinal liquid is added into a glass tube (the inner diameter is 1 cm, the length is 10 cm), and the glass tube is repeatedly rolled on a flat table until the acetone solution is completely volatilized. Soaking cabbage leaves in the liquid medicine with the corresponding concentration for 15 s, drying, placing in a glass tube, inoculating 10-20 heads of Frankliniella occidentalis larvae in the glass tube, repeating the treatment for three times, observing in a constant-temperature observation room after the treatment, investigating the number of live insects after 72 hours, and calculating the mortality.

In this test, Compound I-1, Compound I-3, Compound I-9, Compound I-10, Compound I-13, Compound I-14, Compound I-15, Compound I-16, Compound I-17, Compound I-18, Compound I-21, Compound I-22, Compound I-23, Compound I-25 showed a mortality of more than 90 at 3.25 ppm (3.125 mg/L) for Frankliniella occidentalis larvae, respectively.

According to the method, the compound I-1, the compound I-25, the compound I-10, the compound (X.82) disclosed in CN106045962A and the fluxametamide are selected to be respectively used for carrying out parallel tests on the larvae of the Frankliniella occidentalis. The results of the tests are given in table 2 below.

TABLE 2

| | | Mortality (%) | | |
|---|---|---|---|---|
| Compound | | 6.25 | 3.125 | 1.56 |
| number | Structural formula | ppm | ppm | ppm |
| Compounds I-1 of the invention | 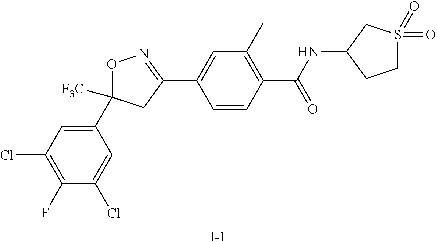 [SMILES: Cc1cc(C2=NOC(c3cc(Cl)c(F)c(Cl)c3)(C(F)(F)F)C2)ccc1C(=O)NC1CCS(=O)(=O)C1 ; [I-]] | 100 | 100 | 100 |
| Compounds I-25 of the invention | 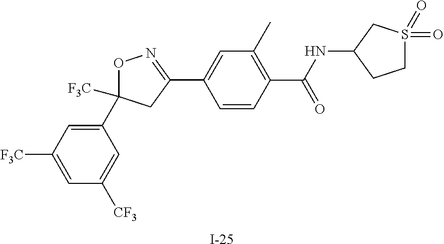 | 100 | 100 | 100 |
| Compounds I-10 of the invention | 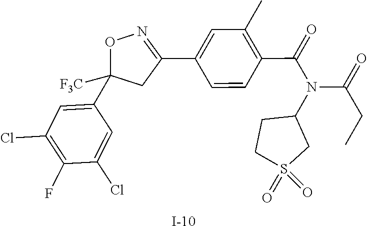 | 100 | 100 | 92.0 |
| Compound disclosed in CN106045962A (X.82) | 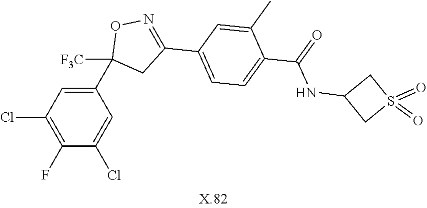 [SMILES: Cc1cc(C2=NOC(c3cc(Cl)c(F)c(Cl)c3)(C(F)(F)F)C2)ccc1C(=O)NC1CS(=O)(=O)C1] | 65.0 | 0 | 0 |
| Fluxametamide | 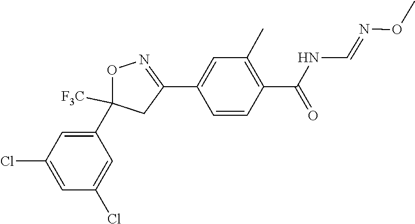 [SMILES: CO/N=C/NC(=O)c1ccc(C2=NOC(c3cc(Cl)cc(Cl)c3)(C(F)(F)F)C2)cc1C] | 0 | 0 | 0 |

| | | |
|---|---|---|
| Blank control | — | 0 |

### Application Example 5

Sensitive Plutella xylostella Controlling Activity Test:

Test targets: Plutella xylostella, 3 rd instar larvae, a sensitive strain raised indoors.

The test method comprises the following steps: selecting fresh cabbage leaves cultivated in a greenhouse, making the cabbage leaves into round with the diameter of 3 cm by using a puncher, putting the circular blade into prepared liquid medicine to be soaked for 10 s according to the sequence from low dose to high dose of experimental design, naturally drying the cabbage leaves in the shade, putting the soaked cabbage leaves into a culture dish with filter paper and the diameter of 9 cm, inoculating regular healthy test insects, n=10, repeating the treatment for 3 times, and additionally setting blank control.

In this test, Compound I-1, Compound I-3, Compound I-9, Compound I-10, Compound I-13, Compound I-14, Compound I-15, Compound I-16, Compound I-17, Compound I-18, Compound I-21, Compound I-22, Compound I-23, Compound I-25 showed a mortality of more than 90 against Plutella xylostella at 1 ppm (1 mg/L), respectively.

According to the method, the compound I-1, the compound I-25, the compound (X.82) disclosed in the patent CN106045962A and the fluxametamide are selected to respectively carry out parallel experiments for killing the Plutella xylostella. The results of the tests are given in table 3 below.

TABLE 3

| | | Mortality (%) | |
|---|---|---|---|
| Compound | | 1 | 0.333 |
| number | Structural formula | ppm | ppm |
| Compounds I-1 of the invention | 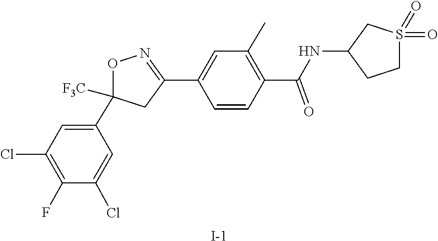 [SMILES: Cc1cc(C2=NOC(c3cc(Cl)c(F)c(Cl)c3)(C(F)(F)F)C2)ccc1C(=O)NC1CCS(=O)(=O)C1 ; [I-]] | 100 | 80.0 |
| Compounds I-25 of the invention | 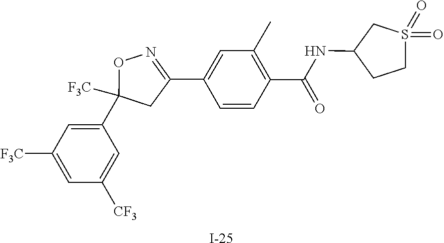 | 100 | 65.0 |
| Compound disclosed in CN106045962A (X.82) | 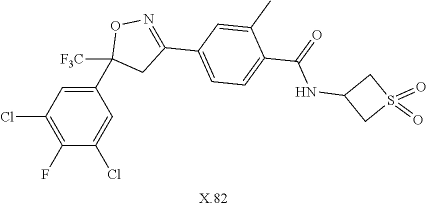 [SMILES: Cc1cc(C2=NOC(c3cc(Cl)c(F)c(Cl)c3)(C(F)(F)F)C2)ccc1C(=O)NC1CS(=O)(=O)C1] | 10.0 | 0 |
| Fluxametamide | 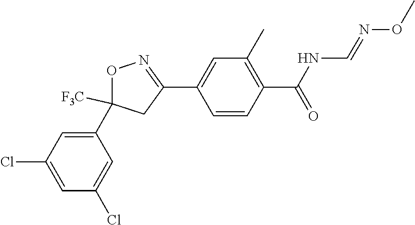 [SMILES: CO/N=C/NC(=O)c1ccc(C2=NOC(c3cc(Cl)cc(Cl)c3)(C(F)(F)F)C2)cc1C] | 5.0 | 0 |

| | | |
|---|---|---|
| Blank control | — | 0 |

As can be seen from the data in table 3, the mortality rate after three days of administration of the compounds I-1, I-25 of the present invention was significantly higher than that of the compound (x.82) and the fluxametamide disclosed in CN106045962A at low concentrations (1 ppm or less). Therefore, the compounds I-1 and I-25 of the invention have higher insecticidal effect on Plutella xylostella when used at lower concentration.

### Application Example 6

Resistant Plutella xylostella Controlling Activity Test:

Leaf dipping method was used, the appropriate amount of radish leaves were dipped into the drug for 10 s, placed in a plastic petri dish padded with filter paper and dried naturally, each dish was connected with 10 heads of 2nd instar small cabbage moths, placed in the observation room at 24° C. and light (16/8 h). 72 h later observation was made, and the body of the insect was touched lightly with a brush and no reaction was regarded as a dead insect, which was repeated for three times, and a blank control was also set up without any additional agent.

And selecting the compound I-1, the compound I-9 and the compound I-25, and carrying out parallel tests for killing resistant Plutella xylostella by contrasting with the fluxametamide and the chlorantraniliprole. The results of the tests are given in Table 4 below.

TABLE 4

| | | Mortality (%) | | |
|---|---|---|---|---|
| Compound | | 5 | 1 | 0.2 |
| number | Structural formula | ppm | ppm | ppm |
| Compounds I-1 of the invention | 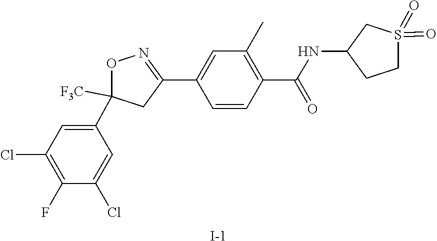 [SMILES: Cc1cc(C2=NOC(c3cc(Cl)c(F)c(Cl)c3)(C(F)(F)F)C2)ccc1C(=O)NC1CCS(=O)(=O)C1 ; [I-]] | 100 | 100 | 100 |
| Compounds I-9 of the invention | 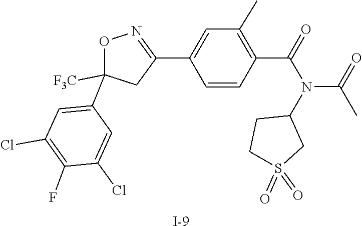 | 100 | 100 | 43.3 |
| Compounds I-25 of the invention | 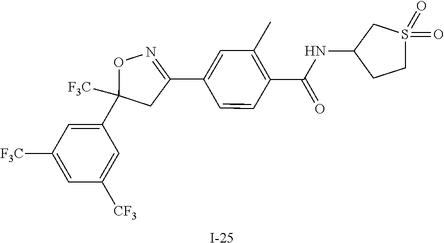 | 100 | 100 | 100 |
| Fluxametamide | 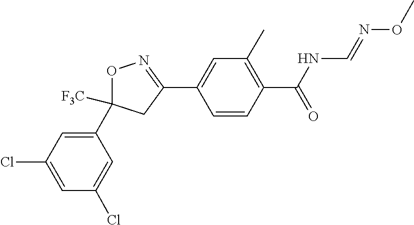 [SMILES: CO/N=C/NC(=O)c1ccc(C2=NOC(c3cc(Cl)cc(Cl)c3)(C(F)(F)F)C2)cc1C] | 100 | 93.3 | 3.33 |
| Chlorantraniliprole | 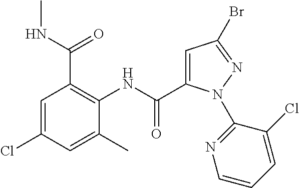 [SMILES: CNC(=O)c1cc(Cl)cc(C)c1NC(=O)c1cc(Br)nn1-c1ncccc1Cl] | 66.67 | 40.00 | 33.33 |

| | | |
|---|---|---|
| Blank control | — | 0 |

As can be seen from the data in Table 4, under very low concentration (0.2 ppm), test by radish leaf infusion method, the death rate of the compounds I-1 and I-25 of the invention to resistant Plutella xylostella reaches 100% after three days of administration, and the compounds have extremely high activity of killing resistant Plutella xylostella, and the activity is obviously higher than that of the marketed insecticides of chlorantraniliprole and fluxaflutolamide. Therefore, the compounds I-1 and I-25 of the invention have high insecticidal effect on resistant Plutella xylostella when used at a very low concentration.

The mortality data of part of the same compounds in the application example 6 in the activity test of the resistant Plutella xylostella is obviously higher than that in the application example 5 in the activity test of the sensitive Plutella xylostella, which is caused by different experimental methods, different experimental conditions, different insect sources and the like, for example, in the application example 5, the cabbage leaves are soaked, in the application example 6, the radish leaves are soaked, the thickness of the cabbage leaves is far larger than that of the radish leaves, and when insects eat food materials with the same weight, the dosage of the compounds eaten by the cabbage leaves is obviously lower, so that the mortality in the different methods in the tests is different.

Preferred embodiments of the present disclosure are described above in detail, but the present disclosure is not limited thereto. Within the technical concept range of the present disclosure, the technical solution of the present disclosure can be subjected to various simple variations, including the combinations of various technical features in any other suitable manner, and these simple variations and combinations should likewise be considered as the contents disclosed by the present disclosure, and all fall within the protection scope of the present disclosure.

1. A compound containing a five-membered heterocycle, wherein the compound has a structure represented by formula (I) or an agriculturally acceptable salt thereof, or a stereoisomer thereof, 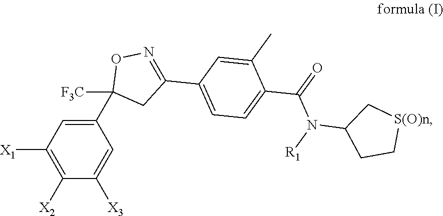
in formula (I),
X₁ and X₃ are each independently F, Cl, Br, I or CF₃;
X₂ is H, F, Cl, Br, I or CF₃; when X₂ is H, X₁ and X₃ are not both Cl;
R₁ is selected from the group consisting of hydrogen, substituted or unsubstituted C₁-C₆ alkyl, C₁-C₄ alkoxy C₁-C₄ alkyl, C₁-C₁₀ alkylcarbonyl, halo C₁-C₁₀ alkylcarbonyl, C₃-C₁₀ cycloalkylcarbonyl, C₃-C₁₀ cycloalkylmethylcarbonyl, C₁-C₁₀ alkoxycarbonyl, halo C₁-C₁₀ alkoxycarbonyl, C₁-C₁₀ alkoxy-C₁-C₄ alkylcarbonyl, halo C₁-C₁₀ alkoxy C₁-C₄ alkylcarbonyl, C₁-C₁₀ alkyl oxalyl, halo C₁-C₁₀ alkyl oxalyl, C₁-C₁₀ alkoxyoxalyl, halo C₁-C₁₀ alkoxyoxalyl, C₁-C₁₀ alkylsulphonyl, halo C₁-C₁₀ alkylsulphonyl, C₁-C₁₀ alkylsulfinyl, halo C₁-C₁₀ alkylsulfinyl, substituted or unsubstituted benzoyl, substituted or unsubstituted phenylsulfonyl, substituted or unsubstituted heteroarylcarbonyl, substituted or unsubstituted C₁-C₆ alkyl, or C₁-C₄ alkoxy C₁-C₄ alkyl;
n is selected from 0, 1 or 2.

2. The compound according to claim 1, wherein X₁, X₂, and X₃ are each independently selected from F, Cl, Br, or CF₃;
R₁ is selected from the group consisting of hydrogen, methyl, ethyl, n-propyl, isopropyl, n-butyl, isobutyl, sec-butyl, tert-butyl, methoxymethyl, methoxyethyl, ethoxymethyl, ethoxyethyl, trifluoromethyl, trichloromethyl, trifluoroethyl or trifluoromethoxy methyl, C₁-C₆ alkylcarbonyl, halo C₁-C₆ alkylcarbonyl, C₃-C₆ cycloalkylcarbonyl, C₃-C₆ cycloalkylmethylcarbonyl, C₁-C₆ alkoxycarbonyl, halo C₁-C₆ alkoxycarbonyl, C₁-C₄ alkoxy C₁-C₂ alkylcarbonyl, halo C₁-C₄ alkoxy C₁-C₂ alkylcarbonyl, C₁-C₆ alkoxalyl, halo C₁-C₆ alkoxalyl, C₁-C₄ alkoxyoxalyl, halo C₁-C₄ alkoxyoxalyl, C₁-C₄ alkylsulfonyl, halo C₁-C₄ alkylsulfonyl, C₁-C₄ alkylsulfinyl, halo C₁-C₄ alkylsulfinyl, halogen substituted or unsubstituted benzoyl, halogen substituted or unsubstituted benzenesulfonyl, carbonyl substituted with a five- or six-membered heteroaromatic ring containing 1-2 heteroatoms selected from O, S or N.

3. The compound according to claim 1, wherein X₁, X₂, and X₃ are each independently selected from F, Cl or CF₃;
R₁ is selected from the group consisting of hydrogen, methyl, ethyl, n-propyl, isopropyl, n-butyl, isobutyl, sec-butyl, tert-butyl, methoxymethyl, ethoxymethyl, ethoxyethyl, acetyl, propionyl, n-butyryl, cyclopropylformyl, methoxyformyl, methoxyacetyl, methoxyacetyl, methoxy oxalyl, ethoxy oxalyl.

4. The compound according to claim 1, wherein the compound is selected from at least one of the compounds represented by the following structures, or an agriculturally acceptable salt thereof, or a stereoisomer thereof: 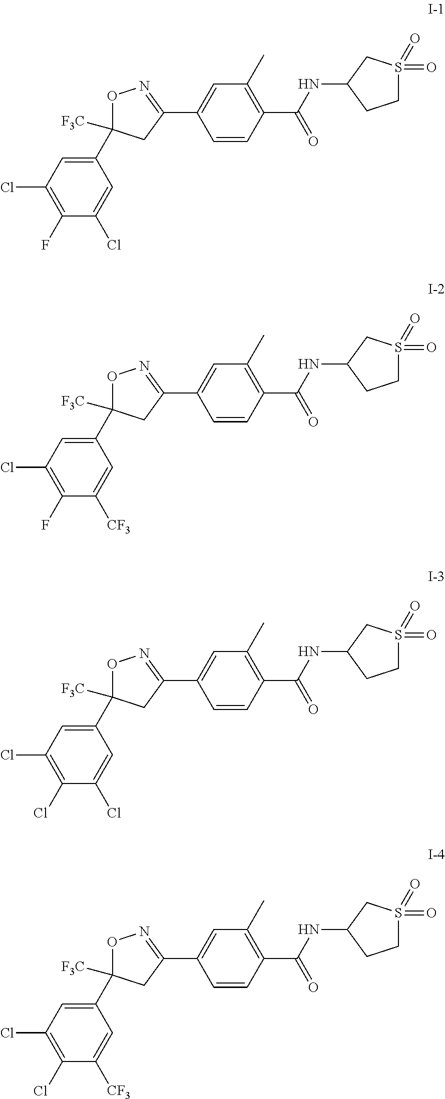 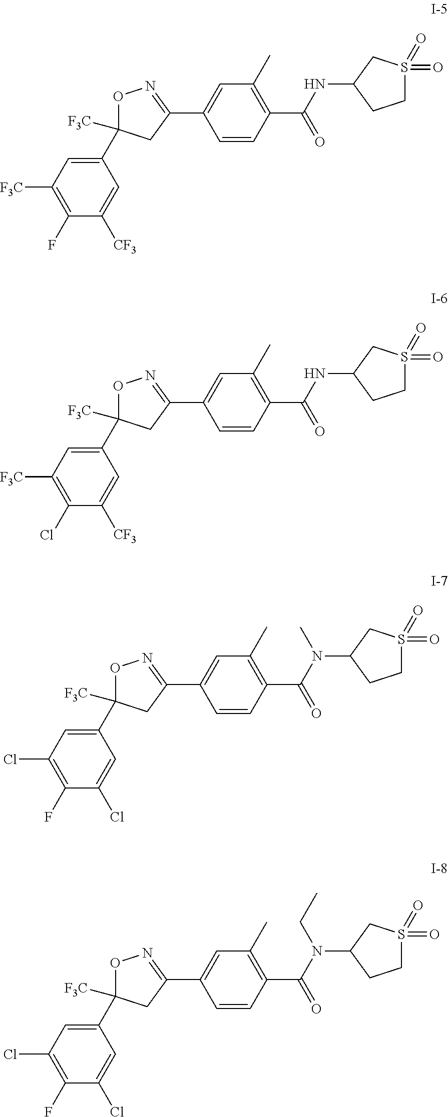 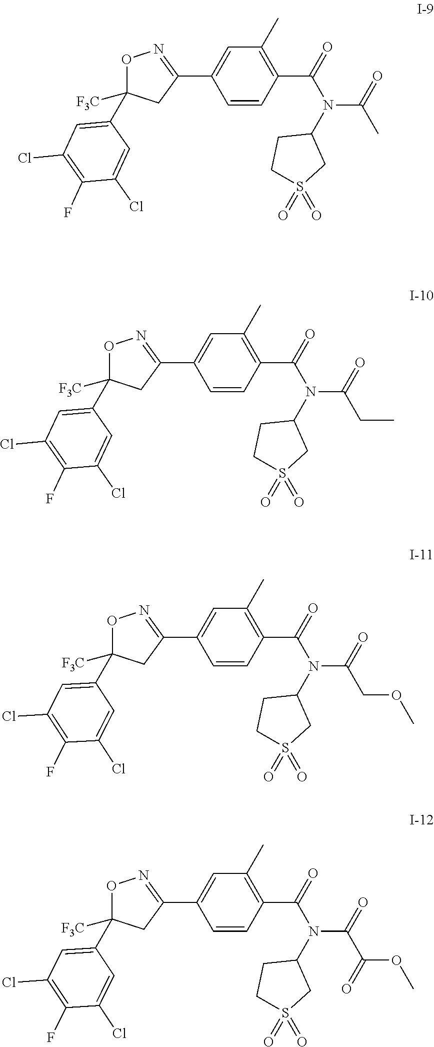 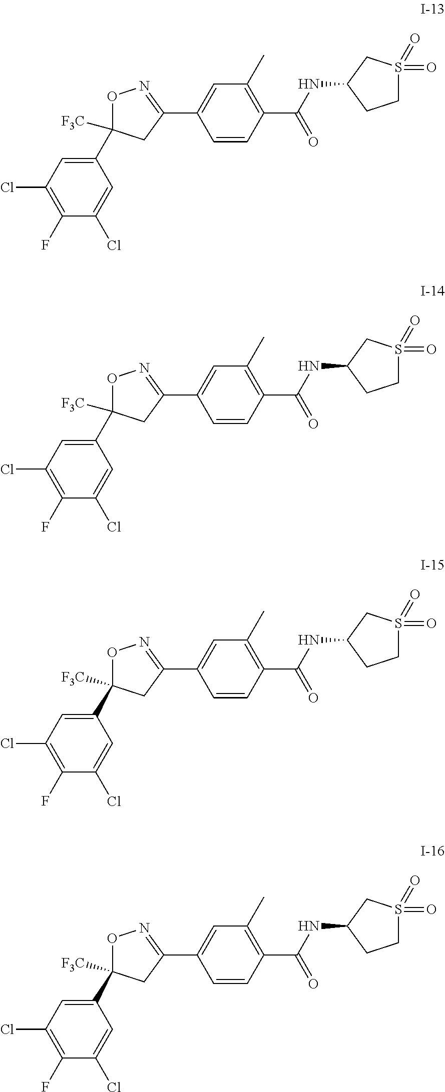 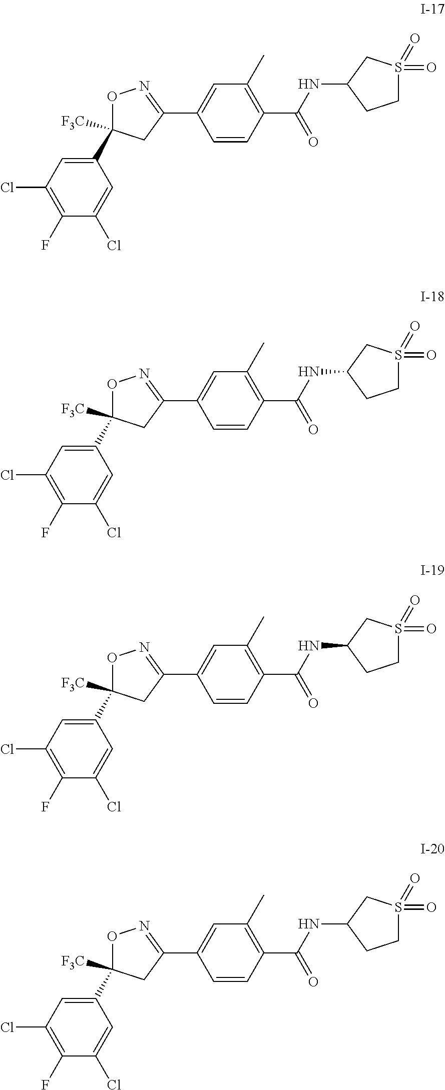 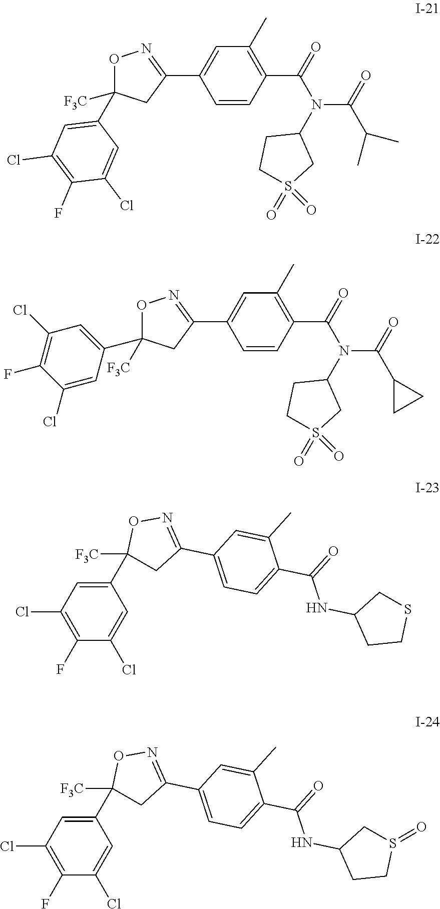 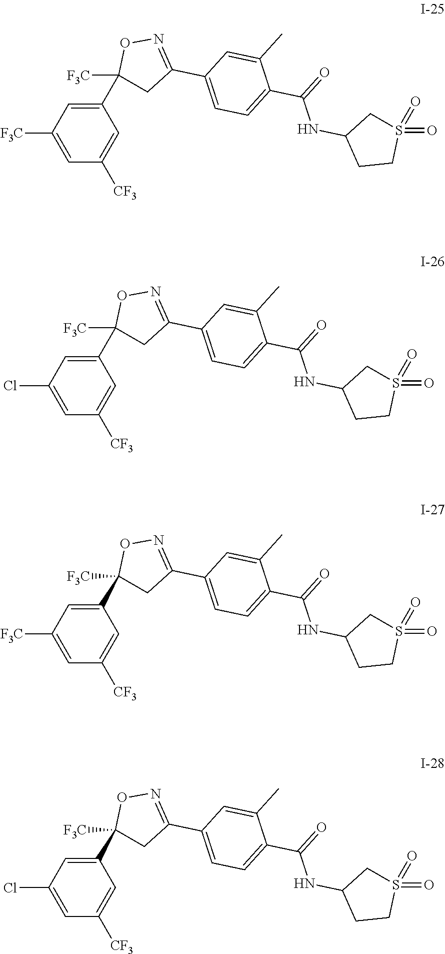 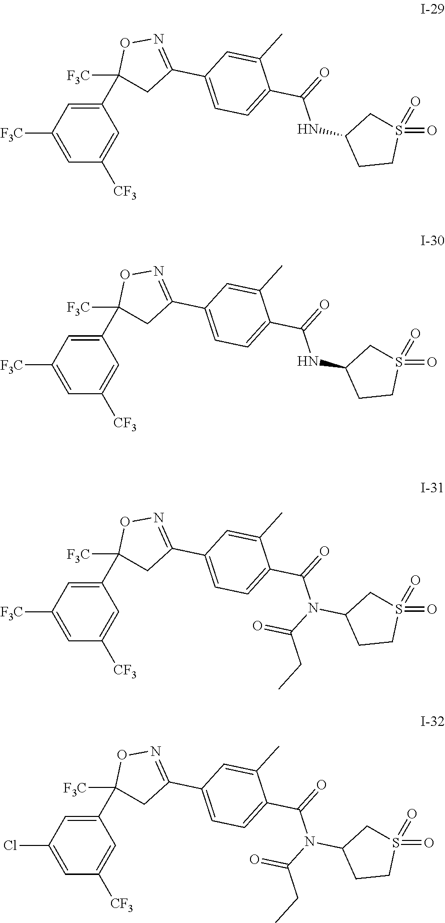
wherein, the compounds I-13, I-14, I-15, I-16, I-17, I-18, I-19 and I-20 are stereoisomers of the compound I-1; compounds I-27, I-29, I-30 are stereoisomers of compounds I-25, and compound I-28 is a stereoisomer of compound I-26.

5. A method of preparing a compound containing a five-membered heterocycle, wherein the method comprises:
(1) compound II and compound III are subjected to a first reaction in a first solvent in the presence of a condensing agent, to obtain compound IV;
(2) compound IV and compound V are subjected to a second reaction in a second solvent in the presence of an alkaline substance, to obtain compound I;
wherein the compound II has a structure represented by formula (II), the compound III has a structure represented by formula (III), the compound IV has a structure represented by formula (IV), the compound V has a structure represented by formula (V), and the compound I has a structure represented by formula (I), 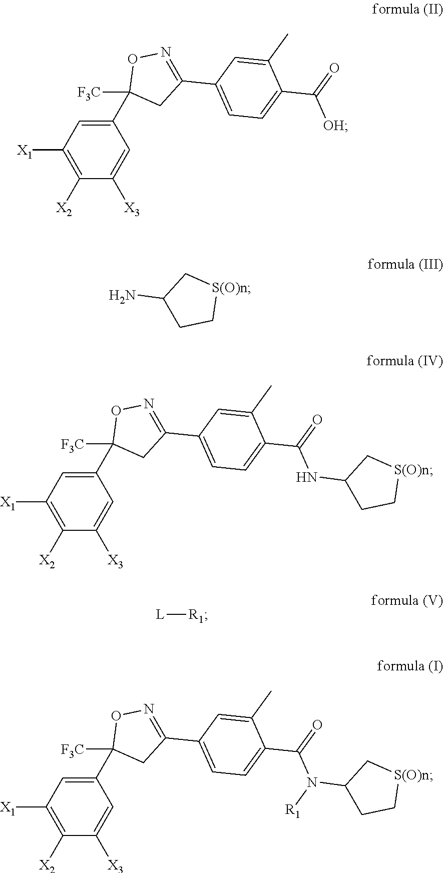
in formula (I), formula (II), formula (III), formula (IV) and formula (V),
X₁ and X₃ are each independently F, Cl, Br, I or CF₃;
X₂ is H, F, Cl, Br, I or CF₃;
when X₂ is H, X₁ and X₃ are not both Cl;
n is selected from 0, 1 or 2;
R₁ is selected from the group consisting of substituted or unsubstituted C₁-C₆ alkyl, C₁-C₄ alkoxy C₁-C₄ alkyl, C₁-C₁₀ alkylcarbonyl, halo C₁-C₁₀ alkylcarbonyl, C₃-C₁₀ cycloalkylcarbonyl, C₃-C₁₀ cycloalkylmethylcarbonyl, C₁-C₁₀ alkoxycarbonyl, halo C₁-C₁₀ alkoxycarbonyl, C₁-C₁₀ alkoxy-C₁-C₄ alkylcarbonyl, halo C₁-C₁₀ alkoxy C₁-C₄ alkylcarbonyl, C₁-C₁₀ alkyl oxalyl, halo C₁-C₁₀ alkyl oxalyl, C₁-C₁₀ alkoxyoxalyl, halo C₁-C₁₀ alkoxyoxalyl, C₁-C₁₀ alkylsulphonyl, halo C₁-C₁₀ alkylsulphonyl, C₁-C₁₀ alkylsulfinyl, halo C₁-C₁₀ alkylsulfinyl, substituted or unsubstituted benzoyl, substituted or unsubstituted phenylsulfonyl, substituted or unsubstituted heteroarylcarbonyl, substituted or unsubstituted C₁-C₆ alkyl, or C₁-C₄ alkoxy C₁-C₄ alkyl;
L is selected from fluorine, chlorine, bromine, iodine, methylsulfonyl, trifluoromethanesulfonyl, phenylsulfonyl or p-toluenesulfonyl.

6. The method according to claim 5, wherein said condensing agent is selected from one or more of dicyclohexylcarbodiimide, 1-(3-dimethylaminopropyl)-3-ethylcarbodiimide hydrochloride, 2-(7-azabenzotriazolyl)-N,N,N′,N′-tetramethyluronium hexafluorophosphate, thionyl chloride, or oxalyl chloride;
and/or, the first solvent and the second solvent are each independently selected from one or more of toluene, xylene, dichloromethane, dichloroethane, acetonitrile, N,N-dimethylformamide, tetrahydrofuran, and acetone;
and/or, the alkaline substance is selected from one or more of triethylamine, diisopropylethylamine, pyridine, N-methylmorpholine, sodium carbonate, potassium carbonate, sodium hydroxide, potassium hydroxide, sodium bicarbonate, potassium bicarbonate, sodium hydride, and sodium amide.

7. (canceled)

8. (canceled)

9. A method of controlling invertebrate pests, wherein said method comprises: applying an insecticidally effective amount of compound as claimed in claim 1, directly or indirectly to an invertebrate pest to be controlled and/or to a medium on which the invertebrate pest grows.

10. The method according to claim 9, wherein said method comprises: treating a plant on which the invertebrate pest grows with an insecticidal effective amount of at least one of the compounds;
and/or, said insecticidal effective amount is 5 g-3000 g per hectare;
and/or, said invertebrate pest are acaridae pests, insects and/or nematodes.
